(19) United States
(12) Patent Application Publication
(10) Pub. No.: US 2024/0018262 A1
(43) Pub. Date: Jan. 18, 2024

(54) ANTIBODY SPECIFIC FOR GPC3 AND USES THEREOF

(71) Applicant: INNOBATION BIO., LTD., Seoul (KR)

(72) Inventors: Ki Tae KIM, Seoul (KR); Seung Koo KIM, Yongin-si (KR)

(73) Assignee: INNOBATION BIO., LTD., Seoul (KR)

(21) Appl. No.: 18/256,369

(22) PCT Filed: Dec. 7, 2021
(86) PCT No.: PCT/KR2021/018470

§ 371 Date: Jun. 7, 2023

(30) Foreign Application Priority Data
Dec. 7, 2020 (KR) 10-2020-0169423

(51) Int. Cl. C07K 16/30; A61K 39/00; C07K 14/725; A61P 35/00; C07K 14/705

(52) U.S. Cl. CPC C07K 16/303; A61K 39/4611; A61K 39/4631; A61K 39/464474; C07K 14/7051; A61P 35/00; C07K 14/70517; C07K 14/70578; C07K 2319/02; C07K 2319/03

(57) ABSTRACT

An antibody specific to GPC3 (Glypican 3) and uses thereof, and more particularly, to an antibody that specifically binds to GPC3, a chimeric antigen receptor comprising the antibody, a CAR-T cell expressing the chimeric antigen receptor, and a pharmaceutical composition for preventing or treating diseases mediated by cells expressing GPC3 comprising the same are provided.

Antibodies that more specifically bound to GPC3 were screened to establish six novel antibodies (10E9, 8B2, 7C6, 7B9, 5E11, 1A8), and it was confirmed that the novel antibodies specifically bound to GPC3 antigen.

Since it was confirmed that GPC3-CAR-T cells prepared in the present invention effectively recognized the GPC3 antigen, induced activation of the CAR-T cells, and effectively killed cells expressing GPC3, GPC3-specific antibody of the present invention, the chimeric antigen receptor prepared using the same and CAR-T cell(s) can be applied to the prevention or treatment of cancers or tumors expressing GPC3.

### TECHNICAL FIELD

The present invention relates to an antibody specific to GPC3 (Glypican 3) and uses thereof, and more particularly, to an antibody that specifically binds to GPC3, a chimeric antigen receptor comprising the antibody, a CAR-T cell expressing the chimeric antigen receptor, and a pharmaceutical composition for preventing or treating diseases mediated by cells expressing GPC3 comprising the same.

### BACKGROUND ART

Glypican 3 (GPC3) is known as a type of oncofetal antigens belonging to the glypican group of glycosyl-phosphatidylinositol-anchored heparin sulfate proteoglycans. It is known that cell membrane-bound glypican-3 consists of two subunits linked by one or more disulfide bonds.

Although expression of GPC3 is not confirmed in adult tissues other than the placenta, expression is confirmed in various cancer tissues such as hepatocellular carcinoma, melanoma, ovarian clear cell carcinoma, and lung squamous cell carcinoma. As described above, GPC3 is classified as a fetal cancer antigen because it is a protein expressed in fetal tissues, similar to proteins such as a-fetoprotein (AFP) and carcinoembryonic antigen (CEA). That is, GPC3 is not expressed in normal tissue cells, but because it exhibits a characteristic of being specifically expressed in cancer cells, it can be used as a target molecule for cancer treatment, a tumor marker, or a diagnostic marker.

Regarding the functions and uses of GPC3, it has been reported that it can function as a hepatocellular carcinoma marker. Recently, it has been reported that GPC3 is expressed in various cancers, especially hepatocellular carcinoma (HCC), melanoma, Wilm's tumor, and hepatoblastoma (Jakubovic and Jothy, Ex Mol Path, 82:184-189,2007; Nakatsura and Nishimura, Biodrugs, 19(2):71-77 2005). It has been reported that tumor treatment such as liver cancer is possible using an antibody against GPC3 (International Publication Patent WO 2014/097648; International Patent Publication No. WO 2018-131586; Korean Patent Publication No. 10-2017-0049831, etc.).

Although several anti-GPC3 antibodies have been reported so far, no antibody has been reported that has satisfactory therapeutic efficacy, particularly excellent therapeutic efficacy for cancer. Therefore, there is a high need for anti-GPC3 antibodies having better therapeutic efficacy.

### DISCLOSURE

### Technical Problem

Therefore, in the present invention, in order to develop an antibody that binds more specifically to GPC3, six novel antibodies (10E9, 8B2, 7C6, 7B9, 5E11, 1A8) were established by screening antibodies that bind to GPC3, and it was confirmed that 6 types of antibodies selected in the present invention specifically bound to the GPC3 antigen. In addition, it was confirmed that it was possible to prepare a chimeric antigen receptor targeting GPC3 and CAR-T cell(s) using the GPC3-specific antibody of the present invention, and the present invention was completed.

Accordingly, an object of the present invention is to provide an antibody that specifically binds to GPC3.

Another object of the present invention is to provide a polynucleotide encoding the antibody, a vector expressing the antibody, and a recombinant cell transformed with the vector.

Another object of the present invention is to provide a pharmaceutical composition for preventing or treating a disease mediated by cells expressing GPC3, including the immune effector cells expressing the antibody or the chimeric antigen receptor targeting GPC3.

Another object of the present invention is to provide a composition for diagnosing or monitoring diseases mediated by cells expressing GPC3 containing the antibody.

### Technical Solution

In order to achieve the above purpose, the present invention is to provide an antibody specifically binding to GPC3 or a fragment thereof, comprising:
(1) a heavy chain variable region including a CDR1 region represented by an amino acid of SEQ ID NO: 1, a CDR2 region represented by an amino acid of SEQ ID NO: 2 and a CDR3 region represented by an amino acid of SEQ ID NO: 3, and a light chain variable region including a CDR1 region represented by an amino acid of SEQ ID NO: 4, a CDR2 region represented by an amino acid of SEQ ID NO: 5 and a CDR3 region represented by an amino acid of SEQ ID NO: 6;
(2) a heavy chain variable region including a CDR1 region represented by an amino acid of SEQ ID NO: 11, a CDR2 region represented by an amino acid of SEQ ID NO: 12 and a CDR3 region represented by an amino acid of SEQ ID NO: 13, and a light chain variable region including a CDR1 region represented by an amino acid of SEQ ID NO: 14, a CDR2 region represented by an amino acid of SEQ ID NO: 15 and a CDR3 region represented by an amino acid of SEQ ID NO: 16;
(3) a heavy chain variable region including a CDR1 region represented by an amino acid of SEQ ID NO: 21, a CDR2 region represented by an amino acid of SEQ ID NO: 22 and a CDR3 region represented by an amino acid of SEQ ID NO: 23, and a light chain variable region including a CDR1 region represented by an amino acid of SEQ ID NO: 24, a CDR2 region represented by an amino acid of SEQ ID NO: 25 and a CDR3 region represented by an amino acid of SEQ ID NO: 26;
(4) a heavy chain variable region including a CDR1 region represented by an amino acid of SEQ ID NO: 31, a CDR2 region represented by an amino acid of SEQ ID NO: 32 and a CDR3 region represented by an amino acid of SEQ ID NO: 33, and a light chain variable region including a CDR1 region represented by an amino acid of SEQ ID NO: 34, a CDR2 region represented by an amino acid of SEQ ID NO: 35 and a CDR3 region represented by an amino acid of SEQ ID NO: 36;
(5) a heavy chain variable region including a CDR1 region represented by an amino acid of SEQ ID NO: 41, a CDR2 region represented by an amino acid of SEQ ID NO: 42 and a CDR3 region represented by an amino acid of SEQ ID NO: 43, and a light chain variable region including a CDR1 region represented by an amino acid of SEQ ID NO: 44, a CDR2 region represented by an amino acid of SEQ ID NO: 45 and a CDR3 region represented by an amino acid of SEQ ID NO: 46; or
(6) a heavy chain variable region including a CDR1 region represented by an amino acid of SEQ ID NO: 51, a CDR2 region represented by an amino acid of SEQ ID NO: 52 and a CDR3 region represented by an amino acid of SEQ ID NO: 53, and a light chain variable region including a CDR1 region represented by an amino acid of SEQ ID NO: 54, a CDR2 region represented by an amino acid of SEQ ID NO: 55 and a CDR3 region represented by an amino acid of SEQ ID NO: 56.

In a preferred embodiment of the present invention, the antibody may be a monoclonal antibody, preferably a single-chain variable fragment (scFv).

In another preferred embodiment of the present invention,
the (1) antibody may comprise a heavy chain variable region represented by an amino acid of SEQ ID NO: 7 and a light chain variable region represented by an amino acid of SEQ ID NO: 8;
the (2) antibody may comprise a heavy chain variable region represented by an amino acid of SEQ ID NO: 17 and a light chain variable region represented by an amino acid of SEQ ID NO: 18;
the (3) antibody may comprise a heavy chain variable region represented by an amino acid of SEQ ID NO: 27 and a light chain variable region represented by an amino acid of SEQ ID NO: 28;
the (4) antibody may comprise a heavy chain variable region represented by an amino acid of SEQ ID NO: 37 and a light chain variable region represented by an amino acid of SEQ ID NO: 38;
the (5) antibody may comprise a heavy chain variable region represented by an amino acid of SEQ ID NO: 47 and a light chain variable region represented by an amino acid of SEQ ID NO: 48; or
the (6) antibody may comprise a heavy chain variable region represented by an amino acid of SEQ ID NO: 57 and a light chain variable region represented by an amino acid of SEQ ID NO: 58.

To achieve another object, the present invention provides a polynucleotide encoding the antibody that specifically binds to GPC3.

In addition, the present invention provides a vector comprising a polynucleotide encoding the antibody that specifically binds to GPC3.

In addition, the present invention provides a recombinant cell transformed with the vector that produces the antibody or a fragment thereof that specifically binds to GPC3.

In order to achieve another object, the present invention provides a chimeric antigen receptor (CAR) comprising: a GPC3-binding domain; a transmembrane domain; a costimulatory domain; and an intracellular signal transduction domain, wherein the GPC3-binding domain may be selected from the antibody specifically binding to GPC3 or a fragment thereof.

The GPC3-binding domain may be selected from the antibody or a fragment thereof capable of specifically binding to GPC3 of the present invention.

In a preferred embodiment of the present invention, the transmembrane domain may be derived from a protein selected from the group consisting of CD8a, CD4, CD28, CD137, CD80, CD86, CD152 and PD1.

In another preferred embodiment of the present invention, the costimulatory domain may be derived from a protein selected from the group consisting of CD28, 4-1BB, OX-40 and ICOS, and the signaling domain may be derived from CD3.

In another preferred embodiment of the present invention, a hinge region located between the C terminus of the GPC3-binding domain and the N terminus of the transmembrane domain may be further included, wherein the hinge region may be derived from CD8a.

In order to achieve another object, the present invention provides a polynucleotide encoding the chimeric antigen receptor (CAR).

In addition, the present invention provides a vector comprising a polynucleotide encoding a chimeric antigen receptor (CAR).

In a preferred embodiment of the present invention, the vector may be a plasmid, a retroviral vector, or a lentiviral vector.

In addition, the present invention provides an immune effector cell expressing the chimeric antigen receptor (CAR) comprising the polynucleotide encoding the chimeric antigen receptor.

In a preferred embodiment of the present invention, the immune effector cell may be a T cell.

In order to achieve another object, the present invention provides a pharmaceutical composition for use in preventing or treating a cancer or tumor expressing GPC3, comprising the antibody specifically binding to GPC3 or the fragment thereof of or the immune effector cell expressing a chimeric antigen receptor targeting GPC3.

In the present invention, the cancer or tumor may be selected from the group consisting of liver cancer, hepatocellular cancer, gastric cancer, breast cancer, lung cancer, ovarian cancer, bronchial cancer, nasopharyngeal cancer, laryngeal cancer, pancreatic cancer, bladder cancer, colon cancer, colon cancer, pancreatic cancer, cervical cancer, brain cancer, prostate cancer, bone cancer, skin cancer, thyroid cancer, parathyroid cancer, kidney cancer, esophageal cancer, biliary tract cancer, testicular cancer, rectal cancer, head and neck cancer, cervical cancer, ureter cancer, osteosarcoma, neuroblastoma, melanoma, fibrosarcoma, rhabdomyosarcoma, astrocytoma, nerve blastoma and glioma.

In addition, the present invention provides a composition for diagnosing or monitoring cancer or tumor expressing GPC3, including an antibody that specifically binds to GPC3.

### Advantageous Effects

In the present invention, antibodies that more specifically bound to GPC3 were screened to establish six novel antibodies (10E9, 8B2, 7C6, 7B9, 5E11, 1A8), and it was confirmed that the novel antibodies specifically bound to GPC3 antigen.

In addition, since it was confirmed that GPC3-CAR-T cells prepared in the present invention effectively recognized the GPC3 antigen, induced activation of the CAR-T cells, and effectively killed cells expressing GPC3, GPC3-specific antibody of the present invention, the chimeric antigen receptor prepared using the same and CAR-T cell(s) can be applied to the prevention or treatment of cancers or tumors expressing GPC3.

### DESCRIPTION OF DRAWINGS

FIG. 1 is data confirming the binding ability of the antibodies (a) 10E9, (b) 8B2, (c) 7C6, (d) 7B9, (e) 5E11, and (f) 1A8 selected in the present invention to GPC3-expressing tumor cells (HepG2) by flow cytometry.

FIG. 2 is a schematic diagram showing a method for producing GPC3-CAR expressing cells using a lentivirus expressing a chimeric antigen receptor (GPC3-CAR) targeting GPC3.

FIG. 3 is a schematic diagram showing a method of transfecting a HEK293 cell line with a lentivirus expressing GPC3-CAR and then confirming the binding ability of the transformed HEK293 cells to the GPC3 peptide.

FIG. 4 is data confirming the expression level of GPC3-CAR and binding ability to GPC3 in HEK293FT cells transfected with a lentiviral vector expressing GPC3-CAR.

FIG. 5 is a schematic diagram showing a lentiviral vector expressing GPC3-CAR and a chimeric antigen receptor expressed in T cells.

FIG. 6 is a schematic view showing a method for preparing GPC3-CART cells using peripheral blood mononuclear cells (PBMC) and a method for confirming the binding ability of the prepared GPC3-CAR-T cells to GPC3 peptides.

FIG. 7 is data confirming the activation of GPC3-CAR-T cells, and confirming the binding ability of GPC3-CAR-CD3⁺T cells, GPC3-CAR-CD4+ T cells and GPC3-CAR-CD8+ T cells to GPC3 peptides.

FIG. 8 is data confirming the activation of GPC3-CAR-T cells, and confirming the degree of IFNy expression by (a) GPC3 (1A8)-CAR-CD4+/CD8⁺T cells, (b) GPC3 (7B9)-CAR-CD4+/CD8⁺T cells, (c) GPC3(10E9)-CAR-CD4+/CD8⁺T cells, (d) GPC3(8B2)-CAR-CD4+/CD8+ T cells and (e) GPC3(5E11) CAR-CD4+/CD8+ T cells in the presence of target cells.

FIG. 9 is data confirming the killing effect of A549 cells (GPC3 non-expressing cells) and HepG2 (GPC3 expressing cells) by GPC3-CAR-T cells.

### Mode of the Invention

Hereinafter, the present invention is described in detail.

Antibodies that specifically bind to GPC3

In one aspect, the present invention relates to an antibody specifically binding to GPC3 or a fragment thereof, comprising:
(1) a heavy chain variable region including a CDR1 region represented by an amino acid of SEQ ID NO: 1, a CDR2 region represented by an amino acid of SEQ ID NO: 2 and a CDR3 region represented by an amino acid of SEQ ID NO: 3, and a light chain variable region including a CDR1 region represented by an amino acid of SEQ ID NO: 4, a CDR2 region represented by an amino acid of SEQ ID NO: 5 and a CDR3 region represented by an amino acid of SEQ ID NO: 6;
(2) a heavy chain variable region including a CDR1 region represented by an amino acid of SEQ ID NO: 11, a CDR2 region represented by an amino acid of SEQ ID NO: 12 and a CDR3 region represented by an amino acid of SEQ ID NO: 13, and a light chain variable region including a CDR1 region represented by an amino acid of SEQ ID NO: 14, a CDR2 region represented by an amino acid of SEQ ID NO: 15 and a CDR3 region represented by an amino acid of SEQ ID NO: 16;
(3) a heavy chain variable region including a CDR1 region represented by an amino acid of SEQ ID NO: 21, a CDR2 region represented by an amino acid of SEQ ID NO: 22 and a CDR3 region represented by an amino acid of SEQ ID NO: 23, and a light chain variable region including a CDR1 region represented by an amino acid of SEQ ID NO: 24, a CDR2 region represented by an amino acid of SEQ ID NO: 25 and a CDR3 region represented by an amino acid of SEQ ID NO: 26;
(4) a heavy chain variable region including a CDR1 region represented by an amino acid of SEQ ID NO: 31, a CDR2 region represented by an amino acid of SEQ ID NO: 32 and a CDR3 region represented by an amino acid of SEQ ID NO: 33, and a light chain variable region including a CDR1 region represented by an amino acid of SEQ ID NO: 34, a CDR2 region represented by an amino acid of SEQ ID NO: 35 and a CDR3 region represented by an amino acid of SEQ ID NO: 36;
(5) a heavy chain variable region including a CDR1 region represented by an amino acid of SEQ ID NO: 41, a CDR2 region represented by an amino acid of SEQ ID NO: 42 and a CDR3 region represented by an amino acid of SEQ ID NO: 43, and a light chain variable region including a CDR1 region represented by an amino acid of SEQ ID NO: 44, a CDR2 region represented by an amino acid of SEQ ID NO: 45 and a CDR3 region represented by an amino acid of SEQ ID NO: 46; or
(6) a heavy chain variable region including a CDR1 region represented by an amino acid of SEQ ID NO: 51, a CDR2 region represented by an amino acid of SEQ ID NO: 52 and a CDR3 region represented by an amino acid of SEQ ID NO: 53, and a light chain variable region including a CDR1 region represented by an amino acid of SEQ ID NO: 54, a CDR2 region represented by an amino acid of SEQ ID NO: 55 and a CDR3 region represented by an amino acid of SEQ ID NO: 56.

In the present invention, the antibody may be a monoclonal antibody. In the present invention, the term “monoclonal antibody” may be an antibody produced by a single antibody-forming cell, with a uniform primary structure (amino acid sequence). It recognizes only one antigenic determinant and is generally produced by culturing a hybridoma cell in which cancer cells and an antibody-producing cell are fused.

The antibody of the present invention is prepared as a humanized antibody with increased similarity to a human antibody by making the remaining parts except for the CDR region, which is a key part for antigen binding, to an amino acid sequence corresponding to an antibody produced by humans. The most common method for humanizing antibody is a CDR-grafting method in which the CDR regions of an animal antibody are grafted into a human antibody, but is not limited thereto, and is known in the art.

As used herein, the term “CDR (complementarity determining region)”, refers to a non-contiguous antigen binding site found within the variable region of both heavy and light chain polypeptides.

In the present invention, the term “antibody” can be used not only in a complete form having two full-length light chains and two full-length heavy chains, but also fragments of antibody molecule. A fragment of antibody molecule means a fragment having at least a peptide tag (epitope) binding function, and includes scFv, Fab, F(ab′), F(a13′) 2, a single domain, etc.

Among antibody fragments, Fab has a structure having variable regions of light and heavy chains, a constant region of light chain and the first constant region of heavy chain (CH1), and has one antigen-binding site. Fab′ differs from Fab in that it has a hinge region comprising one or more cysteine residues at the C terminus of the heavy chain CH1 domain. F(a13′) 2 antibody is produced by forming a disulfide bond with a cysteine residue in the hinge region of Fab′. Fv is a minimal antibody fragment having only a heavy chain variable region and a heavy chain variable region. Recombinant technology for generating an Fv fragment is described in International Patent Publications WO 88/10649, WO 88/106630, WO 88/07085, WO 88/07086 and WO 88/09344. A double chain Fv (dsFv) has a disulfide bond, and a heavy chain variable region and a heavy chain variable region are connected, and a single chain Fv (scFv) is generally connected through a peptide linker, the variable region of the heavy chain and the variable region of the light chain are covalently bonded. Such an antibody fragment can be obtained using a proteolytic enzyme (for example, Fab can be obtained by restriction digestion of the entire antibody with papain, and F(a0 2 fragment can be obtained by digestion with pepsin). Preferably, it can be produced through genetic recombination technology.

The monoclonal antibody that specifically binds to GPC3 of the present invention can be prepared by using all or part of the GPC3 protein as an immunogen (or antigen). More specifically, as an immunogen, GPC3, a fusion protein containing GPC3 protein, or a carrier containing GPC3 protein, if necessary, together with an adjuvant (e.g., Freund adjuvant), is injected once or more by subcutaneous, intramuscular, intravenous, intraperitoneal in mammals except for humans to achieve an immunization. The mammals other than humans are preferably mice, rats, hamsters, malmots, chickens, rabbits, cats, dogs, pigs, goats, sheep, donkeys, horses or cattle (including transgenic animals engineered to produce an antibody from other animals such as mice to produce human antibody), more preferably mouse, rat, hamster, malmot, chicken or rabbit. Antibody-producing cells can be obtained from the immune-sensitized mammal about 1 to 10 days after the final immunization by performing immunization 1 to 4 times every 1 to 21 days from the first immunization. The number of times and intervals for immunization can be appropriately changed depending on the characteristics of the immunogen to be used.

Preparation of a hybridoma secreting a monoclonal antibody can be carried out according to the method of Keira and Mirstein et al. (Nature, 1975, Vol. 256, p. 495-497) and a method similar thereto. Hybridomas can be produced by cell fusion of mammal-derived myeloma cells without autologous antibody-producing ability and antibody-producing cells contained in the group consisting of spleen, lymph node, bone marrow and tonsils, preferably spleen. The mammal may be a mouse, rat, malmot, hamster, chicken, rabbit or human, preferably a mouse, rat, chicken or human.

For cell fusion, for example, a fusion promoter including polyethylene glycol or Sendai virus or a method by electric pulse is used, for example, in a fusion medium containing a fusion promoter, antibody-producing cells and mammalian-derived cells capable of indefinite proliferation. Cells are suspended at a ratio of about 1:1 to 1:10, and in this state, cultured at about 30 to 40° C. for about 1 to 5 minutes. As the fusion medium, for example, MEM medium, RPMI1640 medium, and Iscove's Modified Dulbecco's Medium may be used, and it is preferable to exclude sera such as bovine serum.

In the method of screening the hybridoma clones producing the monoclonal antibody, first, the fusion cells obtained as described above are transferred to a selection medium such as HAT medium, and cultured at about 30 to 40° C. for about 3 days to 3 weeks to kill cells other than hybridomas. Then, after culturing the hybridoma on a microtiter plate, etc., the part with increased reactivity between the immunogen used for the immune response of animals other than humans described above and the culture supernatant was subjected to RIA (radioactive substance-marked immuno antibody) or ELISA (Enzyme-Linked Immunosorbent Assay). The clone producing the monoclonal antibody found above shows specific binding ability to the immunogen.

The monoclonal antibody of the present invention can be obtained by culturing such a hybridoma in vitro or in vivo. For culturing, a conventional method for culturing cells derived from mammals is used, and for collecting monoclonal antibody from a culture or the like, a conventional method in this field for purifying an antibody in general is used. As each method, for example, salting out, dialysis, filtration, concentration, centrifugation, fractional precipitation, gel filtration chromatography, ion exchange chromatography, affinity chromatography, high-performance liquid chromatography, gel electrophoresis or isoelectric point electrophoresis, etc. can be applied, and these are applied in combination as needed. The purified monoclonal antibody is then concentrated and dried to be in a liquid or solid state depending on the use.

In a specific embodiment of the present invention, in order to prepare an antibody that specifically binds to GPC3, hybridomas that produce an anti-GPC3 antibody are prepared and screened, and 7 kinds of antibodies (scFvs) that specifically bind to GPC3 were selected and designated as 10E9, 8B2, 7C6, 7B9, 5E11 and 1A8, respectively.
(1) It was confirmed that 10E9 antibody includes a heavy chain variable region including a CDR1 region represented by an amino acid of SEQ ID NO: 1 (GYTFTSYW), a CDR2 region represented by amino acids of SEQ ID NO: 2 (IYPGNSDT), and a CDR3 region represented by amino acids of SEQ ID NO: 3 (TRGEYDYVLAY), and a light chain variable region including a CDR1 region represented by an amino acid of SEQ ID NO: 4 (QSISNN), a CDR2 region represented by the amino acids of SEQ ID NO: 5 (FAS), and a CDR3 region represented by the amino acids of SEQ ID NO: 6 (QQSNSWPHMYT).

Specifically, 10E9 antibody contained a heavy chain variable region represented by the amino acid of SEQ ID NO: 7 and a light chain variable region represented by the amino acid of SEQ ID NO: 8, wherein the heavy chain variable region was encoded with the nucleotide sequence of SEQ ID NO: 9 and a light chain variable region was encoded with the nucleotide sequence of SEQ ID NO: 10.
(2) It was confirmed that 8B2 antibody comprises a CDR1 region represented by amino acids of SEQ ID NO: 11 (GYTFTSYN), a CDR2 region represented by amino acids of SEQ ID NO: 12 (IYPGNGYT) and a CDR3 region represented by amino acids of SEQ ID NO: 13 (ARGGGPFAY), and a light chain variable region including a CDR1 region represented by an amino acid of SEQ ID NO: 14 (QDVGTA), a CDR2 region represented by amino acids of SEQ ID NO: 15 (WPS), and a CDR3 region represented by amino acids of SEQ ID NO: 16 (QQYSSYPFT).

Specifically, 8B2 antibody contained a heavy chain variable region represented by the amino acid of SEQ ID NO: 17 and a light chain variable region represented by the amino acid of SEQ ID NO: 18, wherein the heavy chain variable region was encoded with the nucleotide sequence of SEQ ID NO: 19 and a light chain variable region was encoded with the nucleotide sequence of SEQ ID NO: 20.
(3) It was confirmed that 7C6 antibody had a heavy chain variable region including a CDR1 region represented by an amino acid of SEQ ID NO: 21 (GYTFTTYY), a CDR2 region represented by amino acids of SEQ ID NO: 22 (INPSNGGT), and a CDR3 region represented by amino acids of SEQ ID NO: 23 (TTFAY), and a light chain variable region including a CDR1 region represented by an amino acid of SEQ ID NO: 24 (QDINKY), a CDR2 region represented by amino acids of SEQ ID NO: 25 (YTS), and a CDR3 region represented by amino acids of SEQ ID NO: 26(LQYDNLWT).

Specifically, the 7C6 antibody contained a heavy chain variable region represented by the amino acid of SEQ ID NO: 27 and a light chain variable region represented by the amino acid of SEQ ID NO: 28, wherein the heavy chain variable region was encoded with the nucleotide sequence of SEQ ID NO: 29 and a light chain variable region was encoded with the nucleotide sequence of SEQ ID NO: 30.
(4) It was confirmed that 7B9 antibody had a heavy chain variable region including a CDR1 region represented by an amino acid of SEQ ID NO: 31 (GYTFTNYW), a CDR2 region represented by amino acids of SEQ ID NO: 32 (IYPGSGST), and a CDR3 region represented by amino acids of SEQ ID NO: 33 (TRGDYDAKFAY), and a light chain variable region including a CDR1 region represented by an amino acid of SEQ ID NO: 34 (SSVSSSY), a CDR2 region represented by amino acids of SEQ ID NO: 35 (STS), and a CDR3 region represented by amino acids of SEQ ID NO: 36 (QQYSGYPLIT).

Specifically, 7B9 antibody contained a heavy chain variable region represented by the amino acid of SEQ ID NO: 37 and a light chain variable region represented by the amino acid of SEQ ID NO: 38, wherein the heavy chain variable region was encoded with the nucleotide sequence of SEQ ID NO: 39 and a light chain variable region was encoded with the nucleotide sequence of SEQ ID NO: 40.
(5) It was confirmed that 5E11 antibody had a heavy chain variable region including a CDR1 region represented by an amino acid of SEQ ID NO: 41 (GYTFSRYW), a CDR2 region represented by amino acids of SEQ ID NO: 42 (ILPGSGST), and a CDR3 region represented by amino acids of SEQ ID NO: 43 (ARSARATYYFDY), and a light chain variable region including a CDR1 region represented by an amino acid of SEQ ID NO: 44 (QDISNY), a CDR2 region represented by amino acids of SEQ ID NO: 45 (YTS), and a CDR3 region represented by amino acids of SEQ ID NO: 46 (QQGNALPYT).

Specifically, 5E11 antibody contained a heavy chain variable region represented by the amino acid of SEQ ID NO: 47 and a light chain variable region represented by the amino acid of SEQ ID NO: 48, wherein the heavy chain variable region was encoded with the nucleotide sequence of SEQ ID NO: 49 and a light chain variable region was encoded with the nucleotide sequence of SEQ ID NO: 50.
(6) It was confirmed that 1A8 antibody had a heavy chain variable region including a CDR1 region represented by an amino acid of SEQ ID NO: 51 (GYTFTSYW), a CDR2 region represented by amino acids of SEQ ID NO: 52 (IYPGSGST), and a CDR3 region represented by amino acids of SEQ ID NO: 53 (TRGHYDYAMDY), and a light chain variable region including a CDR1 region represented by an amino acid of SEQ ID NO: 54 (QNINVW), a CDR2 region represented by amino acids of SEQ ID NO: 55 (KTS), and a CDR3 region represented by amino acids of SEQ ID NO: 56 (QQGQSYPWT).

Specifically, 1A8 antibody contained a heavy chain variable region represented by the amino acid of SEQ ID NO: 57 and a light chain variable region represented by the amino acid of SEQ ID NO: 58, wherein the heavy chain variable region was encoded with the nucleotide sequence of SEQ ID NO: 59 and a light chain variable region was encoded with the nucleotide sequence of SEQ ID NO: 60.

The antibody specific for GPC3 of the present invention is preferably scFv (single chain variable fragment), and can be produced through genetic recombination technology so that the heavy chain variable region and a light chain variable region can be linked with a linker. The linker may preferably be represented by the amino acid sequence of SEQ ID NO: 61 or the nucleotide sequence of SEQ ID NO: 62, but is not limited thereto.

When linked by a light chain variable region-linker-a heavy chain variable region, 10E9 antibody has the amino acid sequence of SEQ ID NO: 63 or the nucleotide sequence of SEQ ID NO: 64, and 8B2 antibody has the amino acid sequence of SEQ ID NO: 65 or the nucleotide sequence of SEQ ID NO: 66, 7C6 antibody has the amino acid sequence of SEQ ID NO: 67 or the nucleotide sequence of SEQ ID NO: 68, and 7B9 antibody has the amino acid sequence of SEQ ID NO: 69 or the nucleotide sequence of SEQ ID NO: 70, 5E11 antibody has the amino acid sequence of SEQ ID NO: 71 or the nucleotide sequence of SEQ ID NO: 72, and 1A8 antibody has the amino acid sequence of SEQ ID NO: 73 or the nucleotide sequence of SEQ ID NO: 74.

In another aspect, the present invention relates to a polynucleotide encoding the antibody that specifically binds to GPC3.

As used herein, the term “polynucleotide” generally refers to a nucleic acid molecule, deoxyribonucleotide or ribonucleotide, or an analog thereof, separated by any length. In some embodiments, a polynucleotide of the present invention can be prepared by (1) in-vitro amplification, such as polymerase chain reaction (PCR) amplification; (2) cloning and recombination; (3) purification such as digestion and gel electrophoretic separation; (4) synthesis such as chemical synthesis, and preferably, the isolated polynucleotide is prepared by recombinant DNA technology. In the present invention, the nucleic acid for encoding the antibody or antigen-binding fragment thereof can be prepared by various methods known in the art, including, but not limited to, restriction fragment operation of synthetic oligonucleotides or application of SOE PCR.

In another aspect, the present invention relates to a vector comprising the polynucleotide encoding the antibody that specifically binds to GPC3, and a recombinant cell transformed with the vector.

In the present invention, the term “vector (expression vector)” refers to a gene preparation including essential regulatory elements such as a promoter so that a target gene can be expressed in an appropriate host cell. A vector may be selected from one or more of a plasmid, a retroviral vector, and a lentiviral vector. Upon transformation into an appropriate host, a vector can replicate and function independently of the host genome, or in some cases can be integrated into the genome itself.

In addition, a vector may contain expression control elements that allow the coding region to be accurately expressed in a suitable host. Such regulatory elements are well known to those skilled in the art and include, for example, promoters, ribosome-binding sites, enhancers and other regulatory elements for regulating gene transcription or mRNA translation. The specific structure of the expression control sequence may vary depending on the function of the species or cell type, but generally contains 5′ non-translated sequence, and a 5′ or 3′ non-translated sequence participating in transcription initiation and translation initiation, respectively, such as TATA box, capped sequence, CAAT sequence, etc. For example, a 5′ non-transcriptional expression control sequence can include a promoter region that can include a promoter sequence for transcription and control of a functionally linked nucleic acid.

As used herein, the term “promoter” means a minimal sequence sufficient to direct transcription. In addition, promoter constructs sufficient to allow expression of a regulatable promoter-dependent gene induced by cell type-specific or external signals or agents may be included, and these constructs may be located in the 5′ or 3′ portion of the gene. Both conservative and inducible promoters are included. Promoter sequences may be derived from prokaryotes, eukaryotes or viruses.

In the present invention, the term “transformant” refers to a cell transformed by introducing a vector having a polynucleotide encoding one or more target proteins into a host cell, and a method for introducing the expression a vector into the host cell to form a transformant are such as a calcium phosphate method or a calcium chloride/rubidium chloride method, an electroporation method, an electroinjection method, a chemical treatment method such as PEG, a method using a gene gun, and the like (Sambrook, J., et al., Molecular Cloning, A Laboratory Manual(2n d ed.), Cold Spring Harbor Laboratory, 1.74, 1989).

When the transformant expressing the vector is cultured in a nutrient medium, an antibody protein can be produced and isolated in large quantities. Medium and culture conditions can be appropriately selected and used depending on the host cell. During culture, conditions such as temperature, medium pH, and culture time should be appropriately adjusted to be suitable for cell growth and mass production of proteins.

The vector according to the present invention can be transformed into a host cell, preferably a mammalian cell, for the production of the antibody. Suitable host cells capable of expressing fully glycosylated proteins include COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells, etc., and these cells are readily available from, for example, ATCC (American Type Culture Collection, USA).

Chimeric antigen receptor targeting GPC3

From another point of view, the present invention also relates to a chimeric antigen receptor (CAR) comprising: a GPC3-binding domain; a transmembrane domain; a costimulatory domain; and an intracellular signal transduction domain,

wherein the GPC3-binding domain is an antibody specifically binding to GPC3 or a fragment thereof, comprising:
(1) a heavy chain variable region including a CDR1 region represented by an amino acid of SEQ ID NO: 1, a CDR2 region represented by an amino acid of SEQ ID NO: 2 and a CDR3 region represented by an amino acid of SEQ ID NO: 3, and a light chain variable region including a CDR1 region represented by an amino acid of SEQ ID NO: 4, a CDR2 region represented by an amino acid of SEQ ID NO: 5 and a CDR3 region represented by an amino acid of SEQ ID NO: 6;
(2) a heavy chain variable region including a CDR1 region represented by an amino acid of SEQ ID NO: 11, a CDR2 region represented by an amino acid of SEQ ID NO: 12 and a CDR3 region represented by an amino acid of SEQ ID NO: 13, and a light chain variable region including a CDR1 region represented by an amino acid of SEQ ID NO: 14, a CDR2 region represented by an amino acid of SEQ ID NO: 15 and a CDR3 region represented by an amino acid of SEQ ID NO: 16;
(3) a heavy chain variable region including a CDR1 region represented by an amino acid of SEQ ID NO: 21, a CDR2 region represented by an amino acid of SEQ ID NO: 22 and a CDR3 region represented by an amino acid of SEQ ID NO: 23, and a light chain variable region including a CDR1 region represented by an amino acid of SEQ ID NO: 24, a CDR2 region represented by an amino acid of SEQ ID NO: 25 and a CDR3 region represented by an amino acid of SEQ ID NO: 26;
(4) a heavy chain variable region including a CDR1 region represented by an amino acid of SEQ ID NO: 31, a CDR2 region represented by an amino acid of SEQ ID NO: 32 and a CDR3 region represented by an amino acid of SEQ ID NO: 33, and a light chain variable region including a CDR1 region represented by an amino acid of SEQ ID NO: 34, a CDR2 region represented by an amino acid of SEQ ID NO: 35 and a CDR3 region represented by an amino acid of SEQ ID NO: 36;
(5) a heavy chain variable region including a CDR1 region represented by an amino acid of SEQ ID NO: 41, a CDR2 region represented by an amino acid of SEQ ID NO: 42 and a CDR3 region represented by an amino acid of SEQ ID NO: 43, and a light chain variable region including a CDR1 region represented by an amino acid of SEQ ID NO: 44, a CDR2 region represented by an amino acid of SEQ ID NO: 45 and a CDR3 region represented by an amino acid of SEQ ID NO: 46; or
(6) a heavy chain variable region including a CDR1 region represented by an amino acid of SEQ ID NO: 51, a CDR2 region represented by an amino acid of SEQ ID NO: 52 and a CDR3 region represented by an amino acid of SEQ ID NO: 53, and a light chain variable region including a CDR1 region represented by an amino acid of SEQ ID NO: 54, a CDR2 region represented by an amino acid of SEQ ID NO: 55 and a CDR3 region represented by an amino acid of SEQ ID NO: 56.

As used herein, the term “chimeric antigen receptor (CAR)” generally refers to a fusion protein containing an extracellular domain having the ability to bind an antigen and one or more intracellular domains. A CAR is a core part of a chimeric antigen receptor T cell (CAR-T) and may contain an antigen binding domain, a transmembrane domain, a co-stimulatory domain, and an intracellular signal transduction domain. A CAR can be combined with a T cell receptor-activating intracellular domain based on the antigen (e.g., GPC3) specificity of the antibody. Genetically modified CAR-expressing T cells can specifically identify and eliminate target antigen-expressing malignant cells.

In the present invention, the term “GPC3-binding domain” generally refers to a domain capable of specifically binding to a GPC3 protein. For example, the GPC3-binding domain may contain an anti-GPC3 antibody or a fragment thereof capable of specifically binding to a human GPC3 polypeptide or a fragment thereof expressed in cancer or tumor cells.

In the present invention, the term “binding domain” can be used interchangeably refers to “extracellular domain”, “extracellular binding domain”, “antigen-specific binding domain” and “extracellular antigen-specific binding domain” and refers to a CAR domain or fragment that has the ability to specifically bind to a target antigen (e.g., GPC3).

In the present invention, anti-GPC3 antibody or a fragment thereof is the aforementioned anti-GPC3 antibody, a monoclonal antibody, preferably a single chain variable fragment (scFv). Specifically, it can be prepared using 10E9, 8B2, 7C6, 7B9, 5E11 and/or 1A8 antibody specific for GPC3 of the present invention.

In the present invention, a signal peptide may be further included at the N-terminus of the GPC3-binding domain, and the “signal peptide” generally refers to a peptide chain for guiding protein transduction. The signal peptide may be a short peptide having a length of 5 to 30 amino acids, preferably represented by the amino acid sequence of SEQ ID NO: 82.

In the present invention, it may further include a hinge region located between the C terminus of the GPC3-binding domain and the N terminus of a transmembrane domain, wherein the hinge region is derived from CD8a, and preferably, it can be represented by the amino acid sequence of SEQ ID NO: 83. The “hinge region” generally refers to the linking region between an antigen-binding region and an immune cell Fc receptor (FcR)-binding region.

In the present invention, “a transmembrane domain” refers to a domain of a CAR that generally passes through a cell membrane and is connected to an intracellular signal transduction domain to play a role in signal transduction. The transmembrane domain may be derived from a protein selected from the group consisting of CD8a, CD4, CD28, CD137, CD80, CD86, CD152 and PD1, and preferably may be represented by the amino acid sequence of SEQ ID NO: 84.

In the present invention, “costimulatory domain” generally refers to an intracellular domain capable of providing immune-stimulatory molecules, which are cell surface molecules necessary for an effective response of lymphocytes to antigens. The costimulatory domain described above may comprise a costimulatory domain of CD28, and may comprise a costimulatory domain of the TNF receptor family, such as the costimulatory domain of OX40 and 4-1BB, preferably it may be 4-1BB represented by the amino acid sequence of SEQ ID NO: 85.

In the present invention, “intracellular signal transduction domain” generally refers to a domain located inside a cell and capable of transmitting a signal. In the present invention, the intracellular signal transduction domain is an intracellular signal transduction domain of the chimeric antigen receptor. For example, the intracellular signal transduction domain may be selected from CD3 intracellular domain, CD28 intracellular domain, CD28 intracellular domain, 4-1BB intracellular domain and OX40 intracellular domain, and preferably it may be CD3 represented by the amino acid sequence of SEQ ID NO: 86.

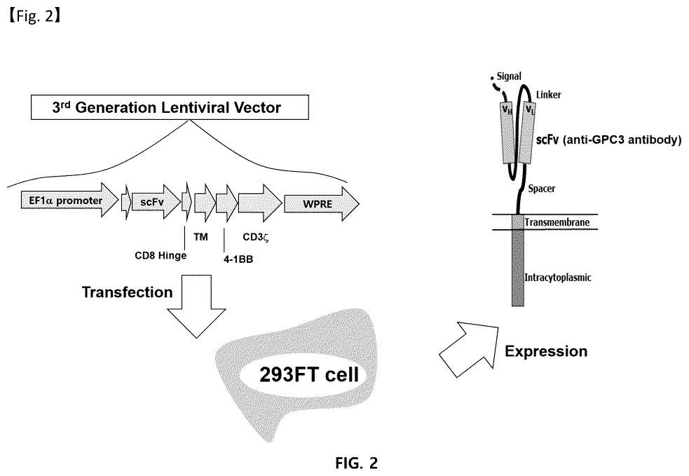

The chimeric antigen receptor targeting GPC3 of the present invention (GPC3-CAR) can be preferably prepared as shown in the schematic diagram shown in FIG. 2.

Polynucleotide Coding Chimeric Antigen Receptor and Vector Expressing Chimeric Antigen Receptor

In another aspect, the present invention relates to a polynucleotide encoding the chimeric antigen receptor (CAR).

In the present invention, the polynucleotide encoding a chimeric antigen receptor (CAR) is a polynucleotide encoding a GPC3-binding domain; a polynucleotide encoding a transmembrane domain; a polynucleotide encoding a costimulatory domain; and a polynucleotide encoding an intracellular signal transduction domain.

A polynucleotide encoding the GPC3-binding domain may be a polynucleotide encoding the antibody specific for GPC3 of the present invention, 10E9, 8B2, 7C6, 7B9, 5E11 and/or 1A8, and a light chain variable region and a heavy chain variable region may be in the form of scFv linked by a linker, and the specific nucleotide sequence may be the same as described above.

Preferably, a polynucleotide encoding a chimeric antigen receptor (CAR) of the present invention may has:

a signal peptide represented by the nucleotide sequence of SEQ ID NO: 76;

10E9 antibody represented by the nucleotide sequence of SEQ ID NO: 64, 8B2 antibody represented by the nucleotide sequence of SEQ ID NO: 66, 7C6 antibody represented by the nucleotide sequence of SEQ ID NO: 68, 7B9 antibody represented by the nucleotide sequence of SEQ ID NO: 70, 5E11 antibody represented by the nucleotide sequence of SEQ ID NO: 72 or 1A8 antibody represented by the nucleotide sequence of SEQ ID NO: 74;
a transmembrane domain represented by the nucleotide sequence of 78;

4-1BB (a costimulatory domain) represented by the nucleotide sequence of SEQ ID NO: 79; and an intracellular signal transduction domain (CD3) represented by the nucleotide sequence of SEQ ID NO: 80.

In addition, a polynucleotide encoding a hinge region may be additionally included between a polynucleotide encoding the GPC3-binding domain and a transmembrane domain, and preferably It may be a CD8 hinge region represented by the nucleotide sequence of SEQ ID NO: 77.

In addition, between the polynucleotide encoding the GPC3-binding domain and the transmembrane domain, a polynucleotide encoding a hinge region may be further included.

In another aspect, the present invention relates to a vector comprising a polynucleotide encoding the chimeric antigen receptor (CAR).

In a specific embodiment of the present invention, the vector is a recombinant virus a vector, preferably a lentivirus vector, and comprises an operably linked EF1a promoter; a polynucleotide encoding a signal peptide; a polynucleotide encoding a GPC3-binding domain; a polynucleotide encoding a transmembrane domain; and a polynucleotide encoding an intracellular signal transduction domain, and may further include a woodchuck hepatitis virus post-transcriptional regulatory element (WPRE) to increase protein expression (refer to FIG. 2).

In addition, the promoter is operably linked to induce expression of an anti-GPC3 antibody (scFv), which is a GPC3-binding domain.

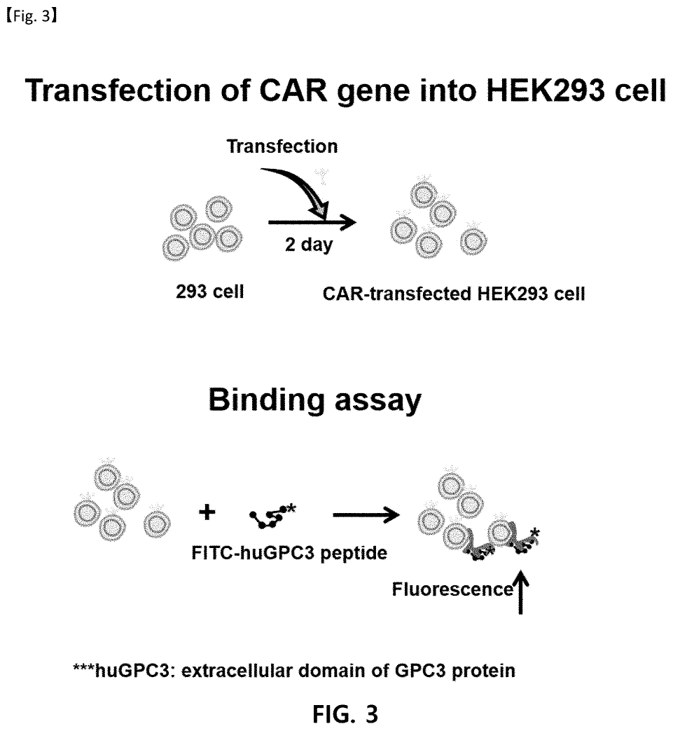

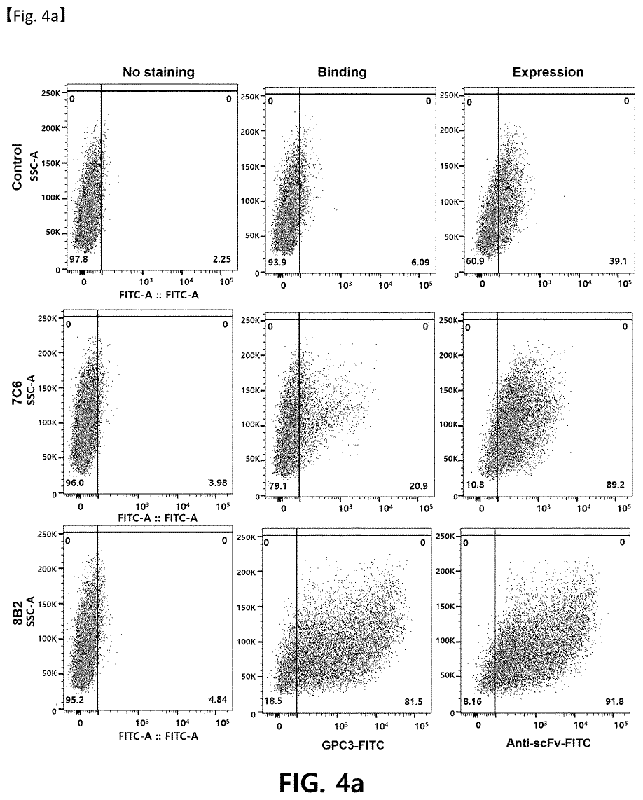

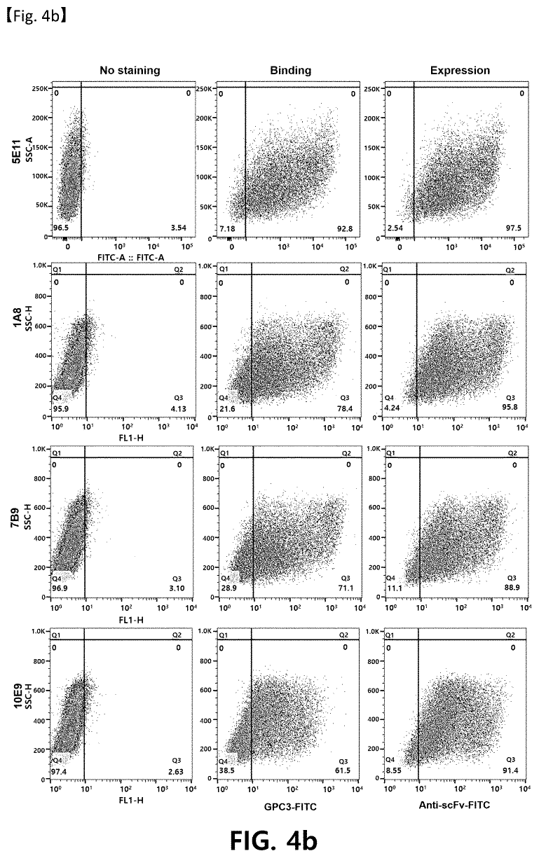

In a specific embodiment of the present invention, as shown in FIGS. 2 and 3, a lentivirus vector into which a polynucleotide encoding GPC3-CAR was inserted was prepared, and as shown in FIG. 4, anti-GPC3 CAR was normally expressed and was confirmed to bind to the GPC3 peptide.

Biological methods for introducing polynucleotides into host cells include the use of DNA and RNA vectors. Viral vectors, and in particular retroviral vectors, have become the most widely used methods for inserting genes into mammalian, e.g., human cells. Other virus vector may be derived from lentiviruses, poxviruses, herpes simplex viruses, adenoviruses and adeno-associated viruses, and the like.

Chemical means for introducing polynucleotides into host cells include colloidal dispersion systems such as macromolecular complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

When a non-viral delivery system is used, an exemplary delivery vehicle is a liposome. The use of lipid preparations is contemplated for the introduction of nucleic acids into host cells (in vitro, ex vivo or in vivo). In another aspect, the nucleic acid may be associated with a lipid. Nucleic acids associated with lipids may be encapsulated within the aqueous interior of the liposome, interspersed within the lipid bilayer of the liposome, attached to the liposome via a linking molecule associated with both the liposome and oligonucleotide, captured within the liposome, complexed with the liposome, dispersed in a lipid-containing solution, mixed with a lipid or combined with a lipid, contained as a suspension in a lipid, contained or complexed with micelles, or otherwise associated with a lipid. Lipid, lipid/DNA or lipid/expression a vector association composition is not limited to any particular structure in solution.

Immune Effector Cell Expressing Chimeric Antigen Receptor (CAR)

In another aspect, the present invention relates to an immune effector cell expressing the chimeric antigen receptor (CAR) includes a vector comprising a polynucleotide encoding a chimeric antigen receptor (CAR), or a polynucleotide encoding a chimeric antigen receptor (CAR).

In the present invention, the immune effector cell may be a mammalian-derived cell, preferably a T cell or a natural killer (NK) cell.

In the present invention, an immune effector cell expressing the chimeric antigen receptor (CAR) can be prepared by introducing the CAR vector of the present invention into an immune effector cell, for example, a T cell or NK cell.

Specifically, CAR vector can be introduced into cells by methods known in the art, such as electroporation, lipofectamine (lipofectamine 2000, Invitrogen), and the like. For example, an immune effector cell can be transformed by a lentiviral vector to integrate the viral genome carrying the CAR molecule into the host genome to ensure long-term and stable expression of the target gene. For another example, a transposon can be used to introduce a CAR transport plasmid and a transferase transport plasmid into a target cell. For another example, a CAR molecule can be added to the genome by a gene editing method (e.g., CRISPRCas9).

An immune effector cell for the production of immune effector cell expressing a chimeric antigen receptor (CAR) can be obtained from a subject, wherein the “subject” includes a living organism (e.g., a mammal from which an immune response can be elicited). Examples of subjects include humans, dogs, cats, mice, rats, and transgenic species thereof. T cells can be obtained from numerous sources, including peripheral blood mononuclear cells, bone marrow, lymph node tissue, umbilical cord blood, thymus tissue, tissue from the site of infection, ascites, pleural effusion, splenic tissue, and tumors.

Such T cells can be obtained from blood units collected from a subject using any of a number of techniques known to those of ordinary skill in the art, for example, Ficoll™ isolation. Cells from blood are obtained by apheresis, and apheresis products typically contain T cells, monocytes, granulocytes, lymphocytes including B cells, other nucleated leukocytes, red blood cells, and platelets.

Cells collected by apheresis can be washed to remove the plasma fraction and place the cells in an appropriate buffer or medium for subsequent processing steps. T cells are isolated from peripheral blood lymphocytes by lysing red blood cells and depleting monocytes, for example by centrifugation through a PERCOLL™ gradient or by countercurrent centrifugation.

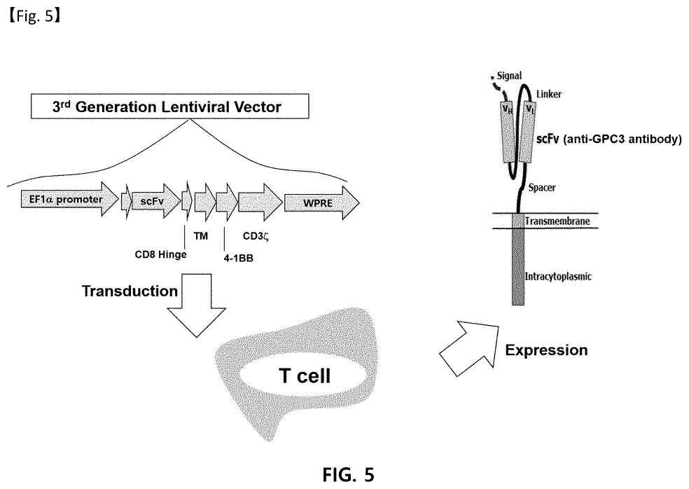

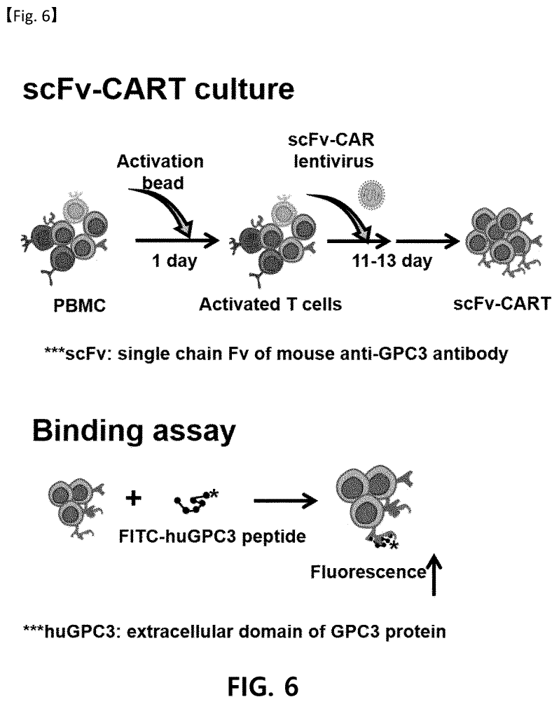

In a specific embodiment of the present invention, as shown in FIGS. 5 and 6, after isolating activated T cells from peripheral blood mononuclear cells (PBMCs), GPC3-CAR lentivirus was transduced into T cells to prepare GPC3-CAR-T cells, and specifically, GPC3-CAR-T cells were prepared using 1A8 antibody, 7B9 antibody, 8B2 antibody, 5E11 antibody, and 10E9 antibody, respectively.

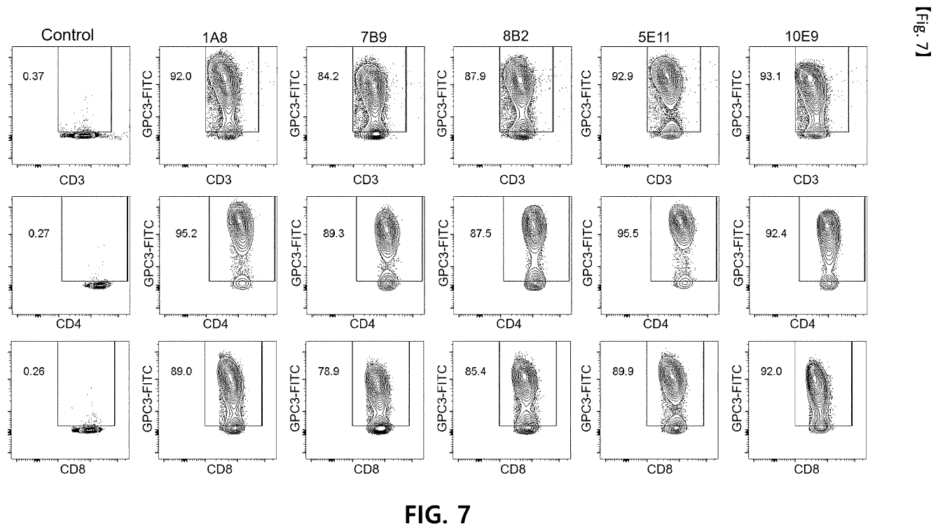

In order to confirm the activity of the prepared GPC3-CAR-T cells, the binding ability to GPC3 peptides for CD3-, CD4-, or CD8-activated GPC3-CAR-T cells was confirmed. As shown in FIG. 7, it was confirmed that the GPC3-CAR-T cells prepared in the present invention bound to the GPC3 peptide.

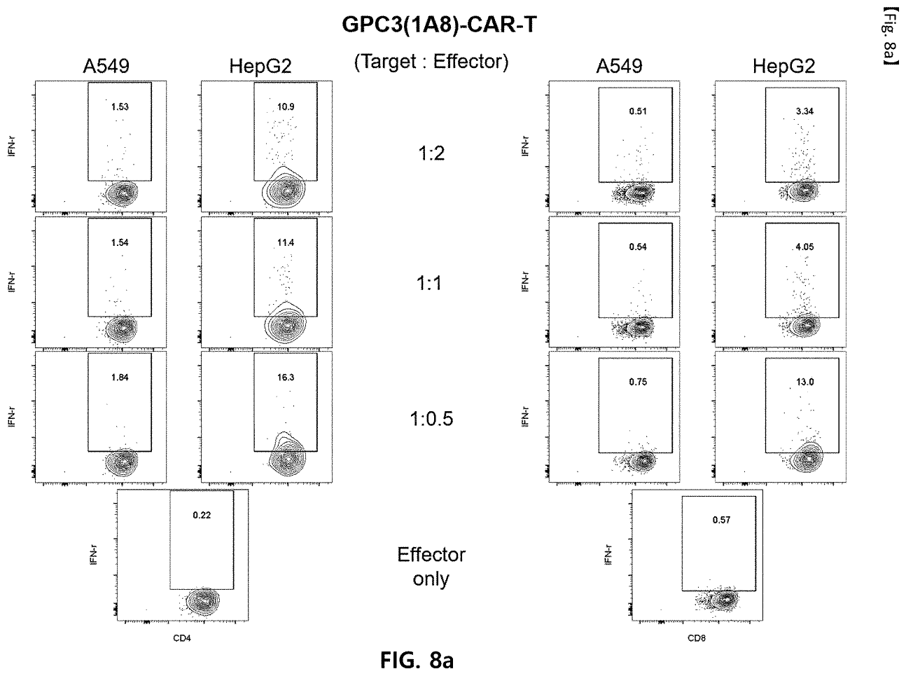

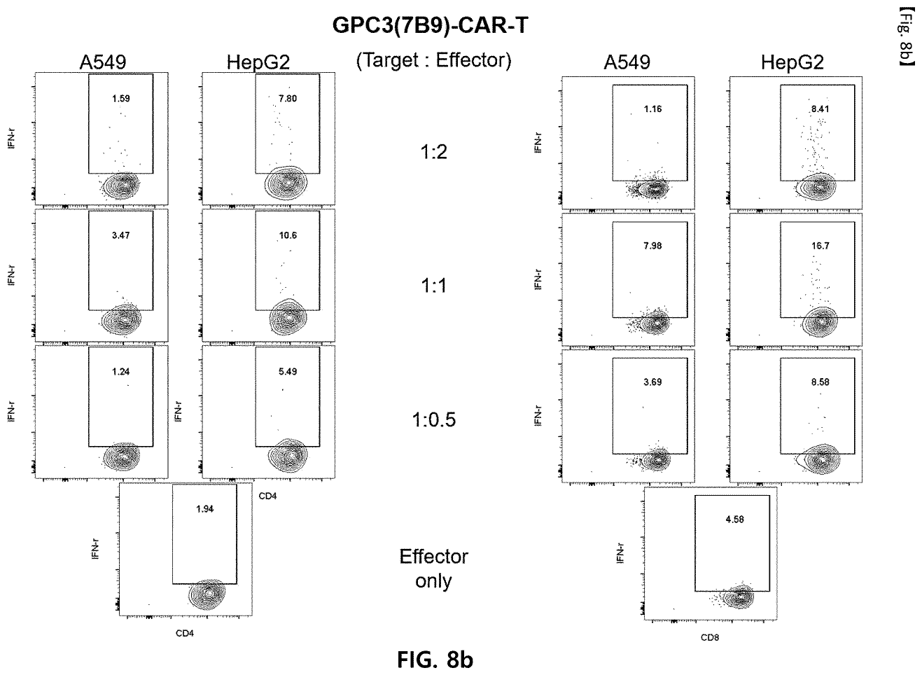

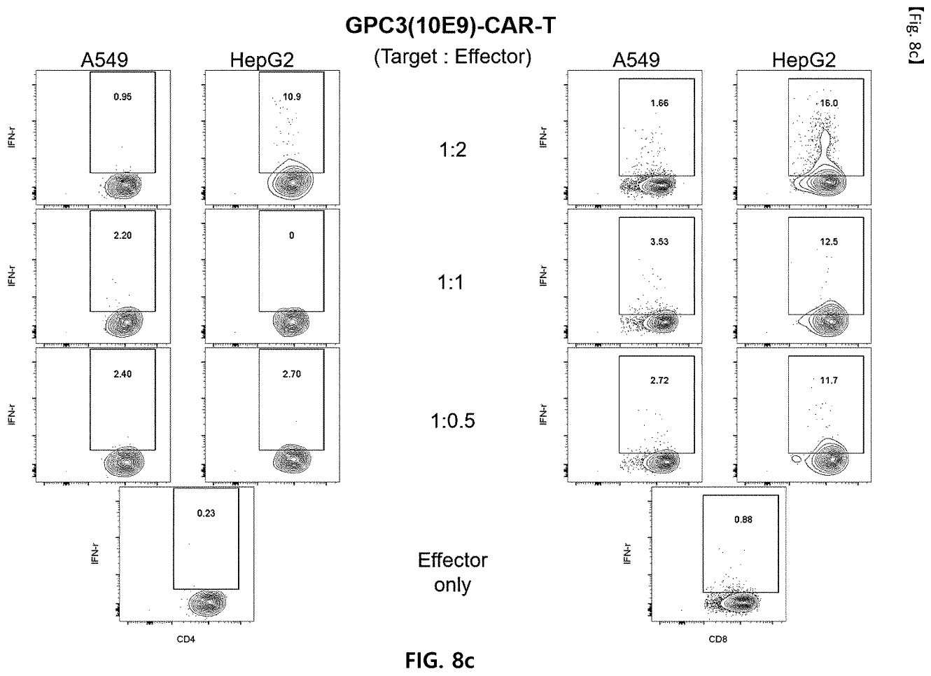

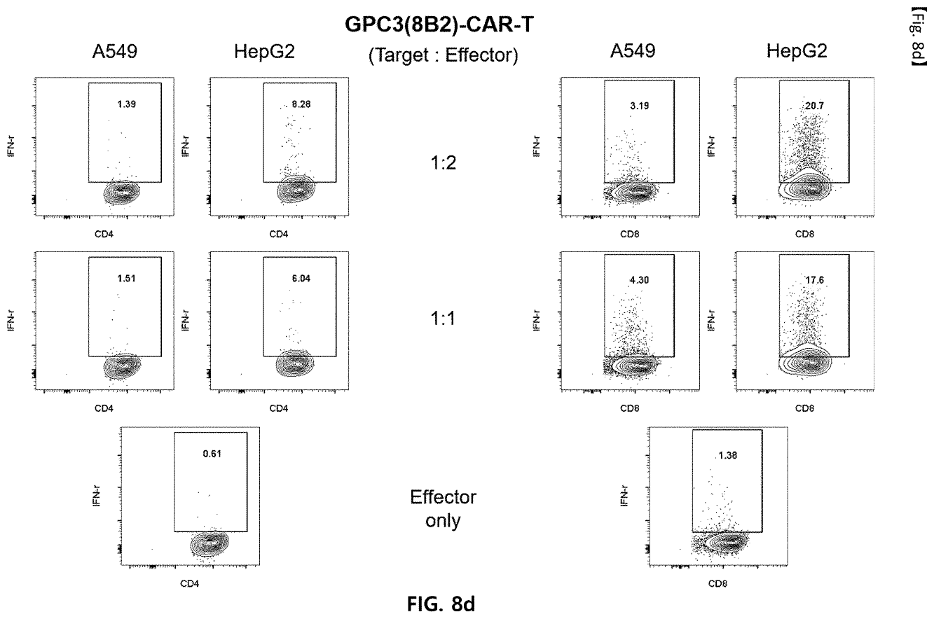

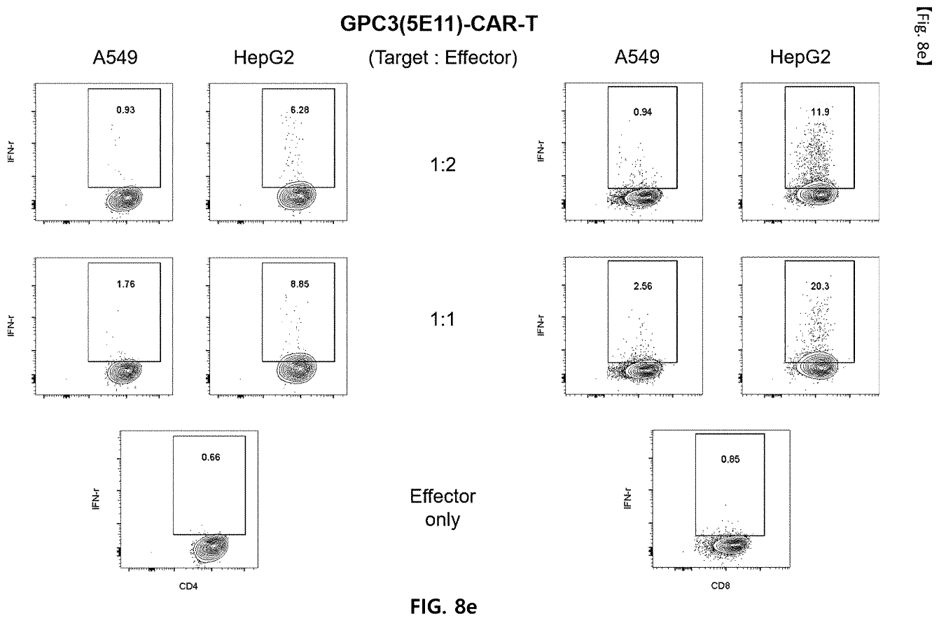

In another specific embodiment of the present invention, in order to confirm activation of GPC3-CAR-T cells, the level of IFNy expression by GPC3-CAR-T cells in the presence of target cells was confirmed. As a result, as shown in FIGS. 8a to 8e, it was confirmed that T cells were not activated in A549 cells that did not express GPC3, whereas T cells were activated in HepG2 cells that expressed GPC3, and IFNy secretion was increased.

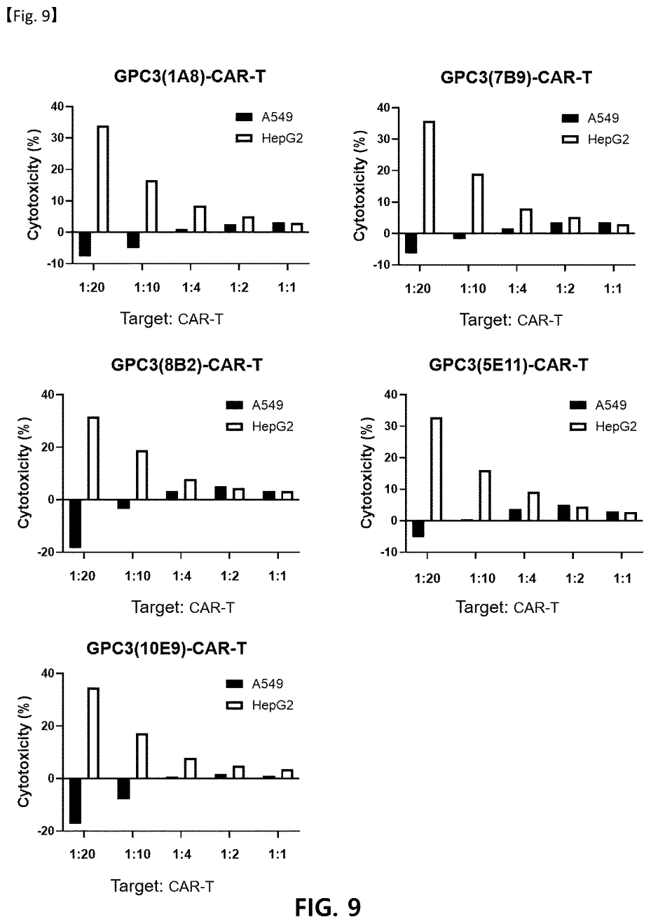

In another specific embodiment of the present invention, as a result of confirming the killing effect of target cells by GPC3-CAR-T cells, as shown in FIG. 9, it was confirmed that the killing effect on HepG2 cells expressing GPC3 was shown.

That is, the chimeric antigen receptor and GPC3-CAR-T cells targeting GPC3 of the present invention can be usefully used as a composition for preventing or treating cancer or tumors expressing GPC3.

Composition for Preventing or Treating Diseases Mediated by GPC3 Expression

In another aspect, the present invention includes a pharmaceutical composition for use in preventing or treating a cancer or tumor expressing GPC3, comprising: the antibody specifically binding to GPC3 or the fragment thereof; or the immune effector cell expressing a chimeric antigen receptor targeting GPC3.

In the present invention, the cancer or tumor expressing GPC3 may be selected from the group consisting of liver cancer, hepatocellular carcinoma, gastric cancer, breast cancer, lung cancer, ovarian cancer, bronchial cancer, nasopharyngeal cancer, laryngeal cancer, pancreatic cancer, bladder cancer, colorectal cancer, colon cancer, pancreatic cancer, cervical cancer, Brain cancer, prostate cancer, bone cancer, skin cancer, thyroid cancer, parathyroid cancer, kidney cancer, esophageal cancer, biliary tract cancer, testicular cancer, rectal cancer, head and neck cancer, cervical cancer, ureter cancer, osteosarcoma, neuroblastoma, melanoma, fibrosarcoma, rhabdomyosarcoma, astrocytoma, neuroblastoma and glioma.

In the present invention, the composition may include a therapeutic agent for a disease mediated by cells expressing GPC3, wherein the therapeutic agent may be covalently bound to the heavy and/or light chain of antibody that specifically binds to GPC3, it can be administered in combination with antibody or GPC3-CAR-T cell specific for GPC3 of the present invention

The therapeutic agent includes a small molecule drug, a peptide drug, a toxin (e.g., a cytotoxin), and the like.

In addition, the therapeutic agent may be an anticancer agent. Anticancer agents reduce the proliferation of cancer cells and include non-peptidyl (i.e., non-protein) compounds, including cytotoxic agents and cytostatic agents. Non-limiting examples of anticancer agents include alkylating agents, nitrosourea, antimetabolites, antitumor antibiotics, plant (vinca)alkaloids, and steroid hormones. Peptide compounds may also be used.

In the pharmaceutical composition, the antibody that specifically binds to GPC3 or an immune effector cell expressing a chimeric antigen receptor targeting GPC3 may be the only active ingredient in the composition for treatment or diagnosis, or, can be used with other active ingredients for example, an anti-T cell, other antibody components such as anti-IFNy or anti-LPS antibody, or non-antibody components such as xanthine.

The pharmaceutical composition preferably contains a therapeutically effective amount of antibody of the present invention. As used herein, the term “therapeutically effective amount” refers to an amount of a therapeutic agent required to treat, ameliorate, or prevent a target disease or condition, or the amount of a therapeutic agent required to exhibit a detectable therapeutic or prophylactic effect. For any antibody, a therapeutically effective dose can be initially determined by cell culture assays or animal models, usually rodents, rabbits, dogs, pigs, or primates. Animal models can also be used to determine appropriate concentration ranges and routes of administration. Such information can be used to determine useful dosages and routes for dosing in humans.

The precise effective amount for a human patient can vary depending on the severity of the disease state, the patient's general health, the patient's age, weight and sex, diet, administration time, administration frequency, drug composition, response sensitivity, and tolerance/response to treatment. The amount can be determined by routine experimentation and is within the scope of the clinician's judgment. In general, an effective dosage is 0.01-50 mg/kg, preferably 0.1-20 mg/kg, more preferably about 15 mg/kg.

The compositions may be administered to the patient individually or in combination with other preparations, agents, or hormones.

The dosage at which the antibody of the present invention is administered depends on the nature of the condition to be treated, the grade of malignant lymphoma or leukemia, and whether the antibody is used to prevent disease or to treat an existing condition.

The frequency of administration depends on the half-life of the antibody molecule and the duration of the drug's effect. If an antibody molecule has a short half-life (e.g., 2 to 10 hours), it may be necessary to provide one or more doses per day. Alternatively, if an antibody molecule has a long half-life (e.g., 2 to 15 days), it may be necessary to provide a dose once a day, once a week, or once every 1 or 2 months.

In addition, the pharmaceutical composition may contain a pharmaceutically acceptable carrier for administration of an antibody. The carrier itself must not cause the production of an antibody that is harmful to the subject receiving the composition, and must be non-toxic. Suitable carriers may be slowly metabolized macromolecules, such as proteins, polypeptides, liposomes, polysaccharides, polylactic acid, polyglycolic acid, amino acid polymers, amino acid copolymers and inactive viral particles.

A pharmaceutically acceptable salt may be used, and it contains for example, mineral acid salts such as hydrochloride, hydrobromide, phosphate and sulfate, or salts of organic acids such as acetic acid, propionic acid, malonic acid and benzoic acid.

A pharmaceutically acceptable carrier in therapeutic compositions may additionally include liquids such as water, saline, glycerol and ethanol. Additionally, auxiliary substances such as wetting agents, emulsifying agents or pH buffering agents may be present in such compositions. The carrier may be formulated as tablets, pills, sugar-coated tablets, capsules, liquids, gels, syrups, slurries and suspensions for ingestion of the pharmaceutical composition by a patient.

Preferred forms for administration may include those suitable for parenteral administration, for example by injection or infusion. When the product is intended for infusion or injection, it may take the form of suspensions, solutions or emulsions in oil or water-soluble excipients, which may contain prescription agents such as suspending, preservative, stabilizing and/or dispersing agents. Alternatively, an antibody molecule may be in anhydrous form and reconstituted with an appropriate sterile solution prior to use.

Once formulated, the compositions of the present invention can be administered directly to a patient. The patients to be treated may be animals. However, the composition is preferably adapted for administration to human patients.

The pharmaceutical composition of the present invention is not limited, but oral, intravenous, intramuscular, intraarterial, intramedullary, intrathecal, intraventricular, transdermal, transcutaneous, subcutaneous, intraperitoneal, intranasal, intestinal, topical, sublingual, intravaginal or rectal routes. Typically, the therapeutic composition may be prepared as injectable forms as liquid solutions or suspensions. In addition, solid forms suitable for solution or suspension in liquid excipients prior to injection may be prepared.

Direct delivery of the composition may generally be achieved by injection, subcutaneous injection, intraperitoneal injection, intravenous injection, intramuscular injection, or may be delivered to the interstitial space of a tissue. In addition, the composition may be administered to the wound site. Dosage treatment may be a single dose schedule or a multiple dose schedule.

The active ingredient in the composition may be an antibody molecule. As such, it may be susceptible to degradation in the gastrointestinal tract. Thus, if the composition is administered by a route using the gastrointestinal tract, the composition may need to contain an agent that protects the antibody from degradation but releases the antibody once absorbed from the gastrointestinal tract.

Diagnosis or Monitoring of Diseases Mediated by Cells Expressing GPC3

In another aspect, the present invention relates to a composition for diagnosing or monitoring a disease mediated by cells expressing GPC3, comprising the antibody that specifically binds to GPC3.

The antibody that specifically binds to GPC3 may be directly or indirectly labeled. An indirect label includes a secondary antibody comprising a detectable label, wherein the secondary antibody binds to an antibody that specifically binds to GPC3. Another indirect label includes biotin, wherein an antibody that specifically binds to biotinylated GPC3 can be detected using avidin or streptavidin containing a detectable label.

A suitable detectable label includes any composition detectable by spectroscopic, photochemical, biochemical, immunochemical, electrical, optical or chemical means. A suitable labels includes, but are not limited to, magnetic beads, fluorescent dyes (e.g., fluorescein isothiocyanate, Texas red, rhodamine, green fluorescent protein, red fluorescent protein, yellow fluorescent protein, etc.), radioactive labels (e.g., For example, 3H, 1251, 35S, 14c or 32P), enzymes (e.g., mustard radish peroxidase, alkaline phosphatase, luciferase and the ones commonly used for enzyme-linked immunosorbent assay (ELISA)) and colorimetric labels such as colloidal gold or tinted glass or plastic (e.g., polystyrene, polypropylene, latex, etc.) beads.

In addition, for diagnosis or monitoring, the antibody may be labeled with a fluorescent protein, and may contain a contrast agent or a radioisotope.

When the antibody that specifically binds to GPC3 of the present invention is used in a diagnostic kit, the antibody is immobilized on a support, and the support may be a microplate, microarray, chip, glass, bead or particle, or a membrane.

Hereinafter, preferred examples are presented to help the understanding of the present invention. However, the following examples are only provided for easier understanding of the present invention, and the contents of the present invention are not limited by the following examples.

### Example 1: Preparation and Selection of the Antibody that Specifically Bound to GPC3

To select the antibody specific for GPC3 peptide, a hybridoma producing the antibody that bound to GPC3 was prepared and the antibody was selected.

First, splenocytes were extracted by immunization with GPC3 protein (Acrobiosystems, cat #GP3-H52H4), and hybridoma cells were prepared through cell fusion with mouse myeloma cells.

Mouse myeloma cells used for cell fusion cannot survive in HAT medium because they do not have HypoxanthineGuanidine-Phosphoribosyl-Transferase (HGPRT), but hybridomas can survive in HAT medium by fusing with splenocytes. Since only hybridomas can be grown using this, they were usually grown in HAT medium until hybridomas were established.

A limiting dilution method was used to select hybridomas producing antibodies binding to GPC3 among the proliferated hybridomas. First, it was made to be less than one cell per 96 well, and then, it was confirmed by ELISA whether the antibody obtained from clones proliferated from one cell bound to GPC3, and clones that bound to GPC3 were selected. The above process was repeated three times to select hybridomas producing the antibody that bound to GPC3. In this way, seven types of antibodies that bound to GPC3 were obtained.

The six types of antibodies were named 10E9, 8B2, 7C6, 7B9, 5E11 and 1A8, respectively, and their nucleotide and amino acid sequences were analyzed. Sequence information for a heavy chain variable region and a light chain variable region of each antibody according to the sequencing result was shown in Tables 1 to 6 below, and the underlined parts in Tables 1 to 6 are complementary determining regions (CDRs).

TABLE 1
Sequence information of 10E9 antibody

| 10E9 | sequence information | SEQ ID NO: |
|---|---|---|
| Heavy chain | GYTFTSYW | SEQ ID NO: 1 |
| variable region | | |
| CDR1 | | |
| Heavy chain | IYPGNSDT | SEQ ID NO: 2 |
| variable region | | |
| CDR2 | | |
| Heavy chain | TRGEYDYVLAY | SEQ ID NO: 3 |
| variable region | | |
| CDR3 | | |
| Light chain | QSISNN | SEQ ID NO: 4 |
| variable region | | |
| CDR1 | | |
| Light chain | FAS | SEQ ID NO: 5 |
| variable region | | |
| CDR2 | | |
| Light chain | QQSNSWPHMYT | SEQ ID NO: 6 |
| variable region | | |
| CDR3 | | |
| Amino acid | EVQLEESGTVLARPGASVKMSCKAS GYTFTSYW | SEQ ID NO: 7 |
| sequence of | MHWIKQRPGQGLEWIGA IYPGNSDT | |
| heavy chain | SYNQKFKGKAKLTAVTSTSTAYMELSSLTNEDSAVYYC | |
| variable region | TRGEYDYVLAY W GQGTLVTVSA | |
| Amino acid | DIVMTQSPATLSVTPGDSVSLSCRAS QSISNN | SEQ ID NO: 8 |
| sequence of light | LHWYQQKSHESPRLLIK FAS | |
| chain variable | QSISGIPSRFSGSGSGTDFTLSINSVETEDFGMYFC | |
| region | QQSNSWPHMYT FGGGTKLEIK | |
| Nucleotide | gaggtgcagctggaggagtctgggactgtgctggcaaggcctggggc | SEQ ID NO: 9 |
| sequence of | ttcagtgaagatgtcctgcaaggcttctggctacacctttaccagctac | |
| heavy chain | tggatgcactggataaaacagaggcctggacagggtctggaatggat | |
| variable region | tggcgctatttatcctggaaatagtgatactagctacaaccagaagttc | |
| | aagggcaaggccaaactgactgcagtcacatccaccagcactgccta | |
| | catggagctcagcagcctgacaaatgaggactctgcggtctattactg | |
| | tacaagaggggaatatgattacgtccttgcttactggggccaagggac | |
| | tctggtcactgtctctgca | |
| Nucleotide | gacattgtgatgacccagtctccagccaccctgtctgtgactccaggag | SEQ ID NO: 10 |
| sequence of light | atagcgtcagtctttcctgcagggccagccaaagtattagcaacaacc | |
| chain variable | tacactggtatcaacaaaaatcacatgagtctccaaggcttctcatca | |
| region | agtttgcttcccagtccatctctgggatcccctccaggttcagtggcagt | |
| | ggatcagggacagatttcactctcagtatcaacagtgtggagactgaa | |
| | gattttggaatgtatttctgtcaacagagtaacagctggcctcacatgt | |
| | acacgttcggaggggggaccaagctggaaataaaa | |

TABLE 2
Sequence information of 8B2 antibody

| 8B2 | sequence information | SEQ ID NO: |
|---|---|---|
| Heavy chain | GYTFTSYN | SEQ ID NO: 11 |
| variable region | | |
| CDR1 | | |
| Heavy chain | IYPGNGYT | SEQ ID NO: 12 |
| variable region | | |
| CDR2 | | |
| Heavy chain | ARGGGPFAY | SEQ ID NO: 13 |
| variable region | | |
| CDR3 | | |
| Light chain | QDVGTA | SEQ ID NO: 14 |
| variable | | |
| region CDR1 | | |
| Light chain | WPS | SEQ ID NO: 15 |
| variable | | |
| region CDR2 | | |
| Light chain | QQYSSYPFT | SEQ ID NO: 16 |
| variable | | |
| region CDR3 | | |
| Amino acid | QVQLQQPGAELVKPGASVKMSCKAS GYTFTSYN | SEQ ID NO: 17 |
| sequence of heavy | MHWVKQTPGQGLEWIGA IYPGNGYT | |
| chain variable | SYNQKFKGKATLTADKSSSTAYMQLSSLTSEDSAVYYC | |
| region | ARGGGPFAY WGQGTLVTVSA | |
| Amino acid | DIVMTQSHKFMSTSVGDRVSITCKAS QDVGTA | SEQ ID NO: 18 |
| sequence of light | VAWYQQKPGQSPKLLIY WPS | |
| chain variable | TRHTGVPDRFTGSGSGTDFTLTISNVQSEDLADYFC | |
| region | QQYSSYPFT FGSGTKLEIK | |
| Nucleotide | caggtccaactgcagcagcctggggctgagctggtgaagcctggg | SEQ ID NO: 19 |
| sequence of heavy | gcctcagtgaagatgtcctgcaaggcttctggctacacatttaccag | |
| chain variable | ttacaatatgcactgggtaaagcagacacctggacagggcctgga | |
| region | atggattggagctatttatccaggaaatggttatacttcctacaatc | |
| | agaagttcaaaggcaaggccacattgactgcagacaaatcctcca | |
| | gcacagcctacatgcagctcagcagcctgacatctgaggactctgc | |
| | ggtctattactgtgcaagaggggggggccgtttgcttactggggcc | |
| | aagggactctggtcactgtctctgca | |
| Nucleotide | gacattgtgatgacccagtctcacaaattcatgtccacatcagtagg | SEQ ID NO: 20 |
| sequence of light | agacagggtcagcatcacctgcaaggccagtcaggatgtgggtac | |
| chain variable | tgctgtagcctggtatcaacagaaaccagggcaatctcctaaacta | |
| region | ctgatttactggccatccacccggcacactggagtccctgatcgctt | |
| | cacaggcagtggatctgggacagatttcactctcaccattagcaat | |
| | gtgcagtctgaagacttggcagattatttctgtcagcaatatagcag | |
| | ctatccattcacgttcggctcggggacaaagttggaaaaaaa | |

TABLE 3
Sequence information of 7C6 antibody

| 7C6 | sequence information | SEQ ID NO: |
|---|---|---|
| Heavy chain | GYTFTTYY | SEQ ID NO: 21 |
| variable region | | |
| CDR1 | | |
| Heavy chain | INPSNGGT | SEQ ID NO: 22 |
| variable region | | |
| CDR2 | | |
| Heavy chain | TTFAY | SEQ ID NO: 23 |
| variable region | | |
| CDR3 | | |
| Light chain | QDINKY | SEQ ID NO: 24 |
| variable | | |
| region CDR1 | | |
| Light chain | YTS | SEQ ID NO: 25 |
| variable | | |
| region CDR2 | | |
| Light chain | LQYDNLWT | SEQ ID NO: 26 |
| variable | | |
| region CDR3 | | |
| Amino acid | QVQLQQPGAELVKPGASVKLSCKAS GYTFTTYY | SEQ ID NO: 27 |
| sequence of heavy | MYWVKQRPGQGLEWIGE INPSNGGT | |
| chain variable | NFNEKFKSKATLTVDKSSSTAYMQLSSLTSEDSAVYYC | |
| region | TTFAY WGQGTLVTVSA | |
| Amino acid | DIVMTQSPSSLSASLGGKVTITCKAS QDINKYI | SEQ ID NO: 28 |
| sequence of light | VWYQHKPGKGPRLLIH YTS | |
| chain variable | ILQPGIPSRFSGSGSGRDYSFSISNLEPEDIATYYC | |
| region | LQYDNLWT FGGGTKLEIK | |
| Nucleotide | caggtccaactgcagcagcctggggctgaactggtgaagcctggg | SEQ ID NO: 29 |
| sequence of heavy | gcttcagtgaagttgtcctgcaaggcttctggctacaccttcaccac | |
| chain variable | ctactatatgtactgggtgaagcagaggcctggacaaggccttgag | |
| region | tggattggagagattaatcctagcaatggtggtactaacttcaatga | |
| | gaagttcaagagcaaggccacactgacggtagacaaatcctccag | |
| | cacagcatacatgcaactcagcagcctgacatctgaggactctgcg | |
| | gtctattactgtacgacgtttgcttactggggccaagggactctggtc | |
| | actgtctctgca | |
| Nucleotide | gacattgtgatgacccagtctccatcctcactgtctgcatctctggga | SEQ ID NO: 30 |
| sequence of light | ggcaaagtcaccatcacttgcaaggcaagccaagacattaacaag | |
| chain variable | tatatagtttggtaccaacacaagcctggaaaaggtcctaggctgc | |

| | |
|---|---|
| region | tcatacattacacatctatattacagccaggcatcccatcaaggttc |
| | agtggaagtgggtctgggagagattattccttcagcatcagcaacc |
| | tggagcctgaagatattgcaacttattattgtctacagtatgataatc |
| | tttggacgttcggtggaggcaccaagctggaaatcaaa |

TABLE 4
Sequence information of 7B9 antibody

| 7B9 | sequence information | SEQ ID NO: |
|---|---|---|
| Heavy chain | GYTFTNYW | SEQ ID NO: 31 |
| variable region | | |
| CDR1 | | |
| Heavy chain | IYPGSGST | SEQ ID NO: 32 |
| variable region | | |
| CDR2 | | |
| Heavy chain | TRGDYDAKFAY | SEQ ID NO: 33 |
| variable region | | |
| CDR3 | | |
| Light chain | SSVSSSY | SEQ ID NO: 34 |
| variable | | |
| region CDR1 | | |
| Light chain | STS | SEQ ID NO: 35 |
| variable | | |
| region CDR2 | | |
| Light chain | QQYSGYPLIT | SEQ ID NO: 36 |
| variable | | |
| region CDR3 | | |
| Amino acid | QVQLQQPGSELVRPGASVKLSCKAS GYTFTNYW | SEQ ID NO: 37 |
| sequence of heavy | MHWVKQRPGQGLEWIGN IYPGSGST | |
| chain variable | NYDEKFKSKATLTVDTSSSTAYMQLSSLTSEDSAVYYC | |
| region | TRGDYDAKFAY WGQGTLVTVSA | |
| Amino acid | DIVMTQSPAIMSASPGEKVTMTCRAS SSVSSSY | SEQ ID NO: 38 |
| sequence of light | LHWYQQKSGASPKLWIY STS | |
| chain variable | KLASGVPARFSGSGSGTSYSLTISSVEAEDAATYYC | |
| region | QQYSGYPLIT FGAGTKVELK | |
| Nucleotide | caggtccaactgcagcagcctgggtctgagctggtgaggcctggag | SEQ ID NO: 39 |
| sequence of heavy | cttcagtgaagctgtcctgcaaggcttctggctacacattcaccaac | |
| chain variable | tactggatgcactgggtgaaacagaggcctggacaaggccttgagt | |
| region | ggattggaaatatttatcctggtagtggtagtactaactacgatgag | |
| | aagttcaagagcaaggccacactgactgtagacacatcctccagc | |
| | acagcctacatgcagctcagcagcctgacatctgaggactctgcgg | |
| | tctattactgtacaagaggggattacgacgcaaagtttgcttactgg | |
| | ggccaagggactctggtcactgtctctgca | |
| Nucleotide | gacattgtgatgacccagtctccagcaatcatgtctgcatctccagg | SEQ ID NO: 40 |
| sequence of light | ggaaaaggtcaccatgacctgcagggccagctcaagtgtaagttcc | |
| chain variable | agttacttgcactggtaccagcagaagtcaggtgcctcccccaaac | |
| region | tctggatttatagcacatccaagttggcttctggagtccctgctcgct | |
| | tcagtggcagtgggtctgggacctcttactctctcacaatcagcagt | |
| | gtggaggctgaagatgctgccacttattactgccagcagtacagtg | |
| | gttacccactcatcacgttcggtgctgggaccaaggtggagctgaa | |
| | a | |

TABLE 5
Sequence information of 5E11 antibody

| 5E11 | sequence information | SEQ ID NO: |
|---|---|---|
| Heavy chain | GYTFSRYW | SEQ ID NO: 41 |
| variable region | | |
| CDR1 | | |
| Heavy chain | ILPGSGST | SEQ ID NO: 42 |
| variable region | | |
| CDR2 | | |
| Heavy chain | ARSARATYYFDY | SEQ ID NO: 43 |
| variable region | | |
| CDR3 | | |
| Light chain | QDISNY | SEQ ID NO: 44 |
| variable | | |
| region CDR1 | | |
| Light chain | YTS | SEQ ID NO: 45 |
| variable | | |
| region CDR2 | | |
| Light chain | QQGNALPYT | SEQ ID NO: 46 |
| variable | | |
| region CDR3 | | |
| Amino acid | QVQLKQSGAELMKPGASVKISCKAT GYTFSRYW | SEQ ID NO: 47 |
| sequence of heavy | IEWVKQRPGHGLEWIGE ILPGSGST | |
| chain variable | SYNEKFKGKATFTADTSSNTAYMQLSSLTSEDSAVYYC | |
| region | ARSARATYYFDY WGQGTTLTVSS | |
| Amino acid | DIQMTQTTSSLSASLGDRVTISCWAS QDISNY | SEQ ID NO: 48 |
| sequence of light | LNWYQQKPDGTVKLLIY YTS | |
| chain variable | TLHSGVPSRFSGSGSGTDYSLTISNLEQEDIATYFC | |
| region | QQGNALPYT FGGGTKLEIK | |
| Nucleotide | caggtgcagctgaagcagtctggagctgagctgatgaagcctggg | SEQ ID NO: 49 |
| sequence of heavy | gcctcagtgaagatatcctgcaaggctactggctacacattcagta | |
| chain variable | ggtactggatagagtgggtaaagcagaggcctggacatggccttg | |
| region | agtggattggagagattttacctggaagtggtagtactagttacaat | |
| | gagaagttcaagggcaaggccacattcactgcagatacatcctcca | |
| | acacagcctacatgcaactcagcagcctgacatctgaggactctgc | |
| | cgtctattactgtgcaagatcagctcgggctacgtactactttgacta | |
| | ctggggccaaggcaccactctcacagtctcctca | |
| Nucleotide | gatatccagatgacacagactacatcctccctgtctgcctctctggg | SEQ ID NO: 50 |
| sequence of light | agacagagtcaccatcagttgctgggcaagtcaggacattagcaat | |
| chain variable | tatttaaactggtatcagcagaaaccagatggaactgttaaactcct | |
| region | gatctactacacatcaacattacactcaggagtcccatcaaggttca | |
| | gtggcagtgggtctggaacagattattctctcaccattagcaacctg | |
| | gagcaagaagatattgccacttacttttgccaacagggtaatgcgc | |
| | ttccgtacacgttcggaggggggaccaagctggaaataaaa | |

TABLE 6
Sequence information of 1A8 antibody

| 1A8 | sequence information | SEQ ID NO: |
|---|---|---|
| Heavy chain | GYTFTSYW | SEQ ID NO: 51 |
| variable region | | |
| CDR1 | | |
| Heavy chain | IYPGSGST | SEQ ID NO: 52 |
| variable region | | |
| CDR2 | | |
| Heavy chain | TRGHYDYAMDY | SEQ ID NO: 53 |
| variable region | | |
| CDR3 | | |
| Light chain | QNINVW | SEQ ID NO: 54 |
| variable | | |
| region CDR1 | | |
| Light chain | KTS | SEQ ID NO: 55 |
| variable | | |
| region CDR2 | | |
| Light chain | QQGQSYPWT | SEQ ID NO: 56 |
| variable | | |
| region CDR3 | | |
| Amino acid | QVQLQQPGSELVRPGASVKLSCKAS GYTFTSYW | SEQ ID NO: 57 |
| sequence of heavy | MHWVKQRHGQGLEWIGN IYPGSGST | |
| chain variable | NYDEKFKSKGTLTVDTSSRTAYMHLSSLTSEDSAVYYC | |
| region | TRGHYDYAMDY WGQGTSVTVSS | |
| Amino acid | DIVMTQSPSSLSASLGDTITITCHAS QNINVW | SEQ ID NO: 58 |
| sequence of light | LSWYQQKPGNIPKLLIY KTS | |
| chain variable | NLHTGVPSRFSGSGSGTGFTLTISSLQPEDIATYYC | |
| region | QQGQSYPWT FGGGTKLEIK | |
| Nucleotide | caggtccaactgcagcagcctgggtctgagctggtgaggcctggag | SEQ ID NO: 59 |
| sequence of heavy | cttcagtgaagctgtcctgcaaggcttctggctacacattcaccagc | |
| chain variable | tactggatgcactgggtgaagcagaggcatggacaaggccttgag | |
| region | tggattggaaatatttatcctggtagtggtagtactaactacgatga | |
| | gaagttcaagagcaagggcacactgactgtagacacatcctccag | |
| | aacagcctacatgcacctcagcagcctgacatctgaggactctgcg | |
| | gtctattactgtacaaggggtcactacgactatgctatggactactg | |
| | gggtcaaggaacctcagtcaccgtctcctca | |
| Nucleotide | gacattgtgatgacccagtctccatccagtctgtctgcatcccttgga | SEQ ID NO: 60 |
| sequence of light | gacacaattaccatcacttgccatgccagtcagaacattaatgtttg | |
| chain variable | gttaagctggtaccagcagaaaccaggaaatattcctaaactattg | |
| region | atctataagacttccaacttgcacacaggcgtcccatcaaggtttag | |
| | tggcagtggatctggaacaggtttcacattaaccatcagcagcctg | |
| | cagcctgaagacattgccacttactactgtcaacagggtcaaagtt | |
| | atccgtggacgttcggtggaggcaccaagctggaaatcaaa | |

### Example 2: Confirmation of Specificity of the Selected Antibody for GPC3-ELISA Analysis

In the present invention, in order to confirm the specificity for GPC3 of 10E9, 8B2, 7C6, 7B9, 5E11 and 1A8 antibodies established in Example 1, ELISA analysis was performed.

First, in order to encode the GPC3 peptide, GPC3 protein (Acrobiosystems, cat #GP3-H52H4) was dispensed into a 96-well plate at an amount of 100 newell and reacted overnight at 4° C. Then, after treatment with 1 X PBST containing 3% BSA, it was blocked for 30 minutes at room temperature.

100 μl of hybridoma cell culture of each clone producing 10E9, 8B2, 7C6, 7B9, 5E11 or 1A8 antibody was treated in each well, reacted at room temperature for 2 hours, and then washed three times with 1 X PBST. The secondary antibody (anti-HRP, 1:10,000) was treated and reacted at room temperature for 30 minutes, washed three times with 1 X PBST, and then treated with TMB for color development and reacted at room temperature for 5 minutes. Finally, the reaction was terminated by treatment with a stop solution of 1N H2SO 4, and then the absorbance was measured at 450 nm.

TABLE 7
ELISA experimental conditions

| | |
|---|---|
| ELISA reader | Infinite F50 |
| Measurement Filter | 450 nm |
| Measurement Mode | Single Point Photo |
| Antigen Coating | 100 ng/well |
| 2nd Antibody (Anti-mIgG-HRP) | 1:10,000 dilution |
| Substrate | TMB |

TABLE 8
ELISA experiment results

| antibody type | OD450 measurement |
|---|---|
| 1A8 | 2.653 |
| 5E11 | 0.942 |
| 7B9 | 2.540 |
| 7C6 | 2.882 |
| 8B2 | 1.013 |
| 10E9 | 2.317 |

As a result, as shown in Table 8, it was confirmed that all of the antibodies selected in the present invention specifically bound to GPC3.

### Example 3: Confirmation of specificity of the selected antibody to GPC3-flow cytometer

In the present invention, in order to confirm the specificity for GPC3 of 10E9, 8B2, 7C6, 7B9, 5E11 and 1A8 antibodies established in Example 1, flow cytometry was performed.

First, hepatoma cell line HepG2 expressing GPC3 was reacted with 1 μg of each of 10E9, 8B2, 7C6, 7B9, 5E11, and 1A8 antibodies for 30 minutes, and then, after staining the surface with a secondary antibody, it was measured by flow cytometry.

GPC3 antibody (Sino biology Glypican3 APC cat #100393-R024-A, 10 u.1) was used as a positive control, and PE-conjugated anti-mouse IgG antibody (PE-conjugated goat anti-mouse IgG; Biolegend Inc., cat #405307, USA, 5 μl) was used as a secondary antibody.

TABLE 9
Flow cytometry results

| Antibody type | Count | Median | Mean |
|---|---|---|---|
| 10E9 | 3141 | 25208 | 33048 |
| 8B2 | 3218 | 31407 | 40046 |
| 7C6 | 3255 | 61751 | 81403 |
| 7B9 | 3269 | 21956 | 27346 |
| 5E11 | 3404 | 40223 | 52602 |
| 1A8 | 3193 | 29019 | 36561 |
| 2nd Ab alone | 3283 | 205 | 243 |

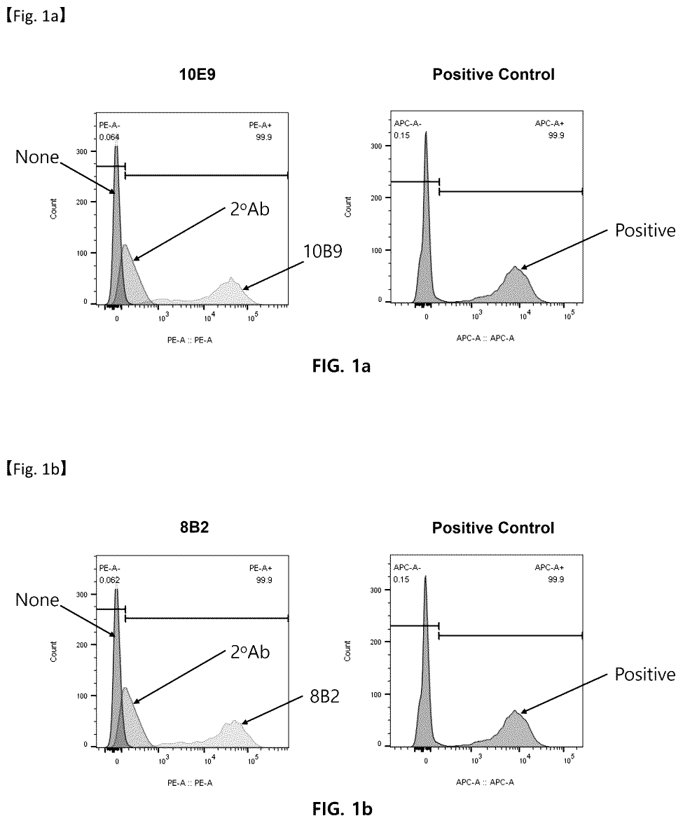

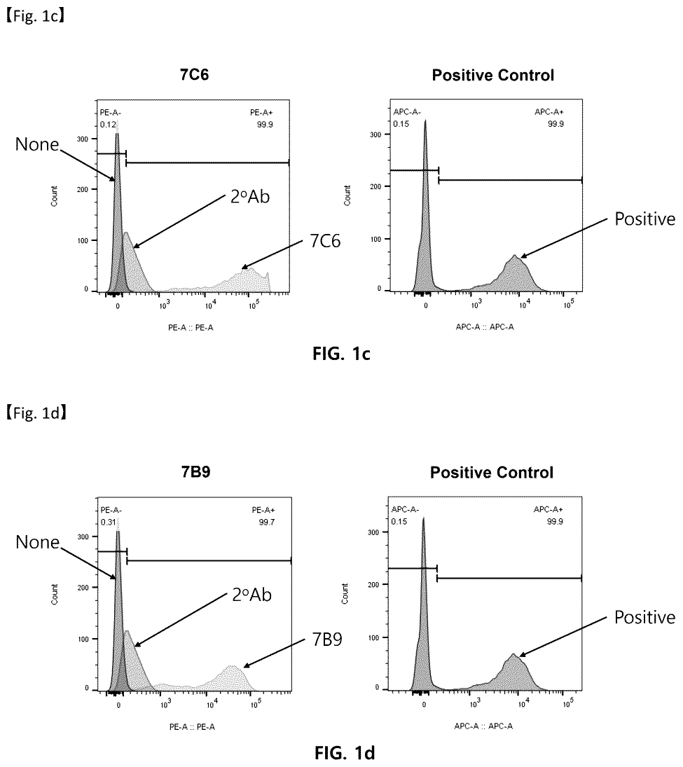

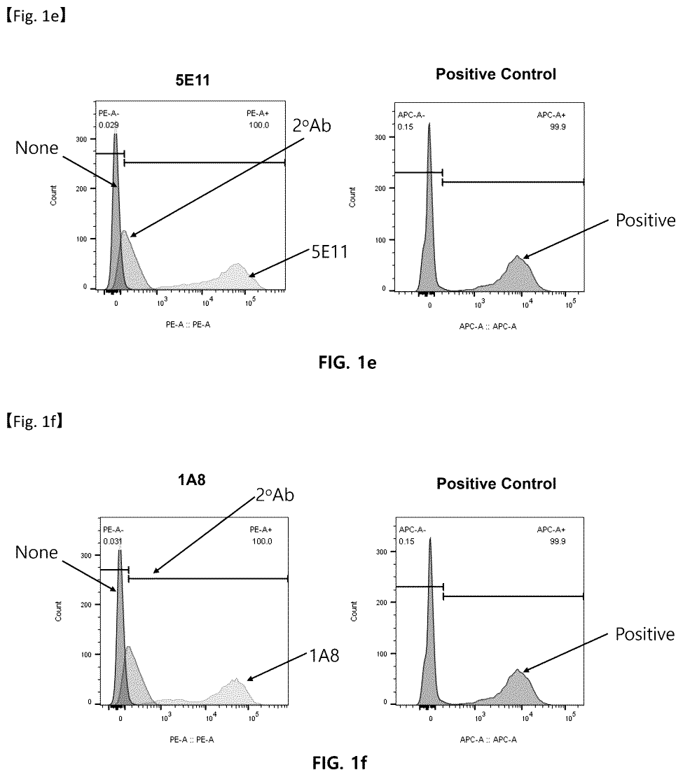

As a result, as shown in FIG. 1 and Table 9, it was confirmed that all of the antibodies 10E9, 8B2, 7C6, 7B9, 5E11 and 1A8 specifically bound to cells expressing GPC3.

Since the antibody selected in the present invention specifically recognized GPC3-expressing cells, it can effectively induce cytotoxicity or death by immune cells/macrophages by inhibiting immune evasion of cancer or tumor cells expressing GPC3. Therefore, the anti-GPC3 antibody of the present invention can be usefully utilized as a composition for preventing or treating cancer or tumor expressing GPC3.

### Example 4: Vector Construction Expressing Chimeric Antigen Receptor Targeting GPC3 (GPC3-CAR)

In the present invention, a lentivirus vector (GPC3-CAR lentivirus) expressing a chimeric antigen receptor (CAR) targeting GPC3 was prepared using the GPC3 antibody prepared in Example 1.

As shown in the schematic diagram of FIG. 2, DNA of CAR composed of:
EF1α promoter (SEQ ID NO: 75);
a polynucleotide encoding a signal peptide (SEQ ID NO: 76);
a polynucleotide encoding the GPC3-binding domain (10E9 antibody represented by the nucleotide sequence of SEQ ID NO: 64, 8B2 antibody represented by the nucleotide sequence of SEQ ID NO: 66, 7C6 antibody represented by the nucleotide sequence of SEQ ID NO: 68, 7B9 antibody represented by the nucleotide sequence of SEQ ID NO: 70, 5E11 antibody represented by the nucleotide sequence of SEQ ID NO: 72, or 1A8 antibody represented by the nucleotide sequence of SEQ ID NO: 74);
a polynucleotide encoding the CD8 hinge region (SEQ ID NO: 77);
a polynucleotide encoding a transmembrane domain (SEQ ID NO: 78);
a polynucleotide encoding 4-1BB (co-stimulatory domain) (SEQ ID NO: 79);
a polynucleotide encoding CD3 (intracellular signaling domain) (SEQ ID NO: 80); and
a polynucleotide encoding WPRE (SEQ ID NO: 81);
was synthesized in vitro and inserted into a third-generation lentiviral vector.

Lentiviral vector DNA (0.5 pg) was transferred to HEK293FT cells (5×10⁵ cells/500 p.1), and 293HEK cells expressing the GPC3-CAR gene were constructed. Lipofectamine 3000 transfection kit (Invitrogen, cat #L3000-015) was used to transfer the gene to 293HEK cells, and cultured in Opti-MEM (gibco, cat #51985-034) medium for 4 hours.

As a result of confirming through flow cytometry that the GPC3-specific CAR was normally expressed and bound to the GPC3 peptide in HEK293FT transformed with the lentiviral vector DNA (FIG. 3), as shown in FIG. 4, GPC3-CAR was normally expressed and confirmed to bind to GPC3 peptide. Accordingly, anti-GPC3 antibodies 10E9, 8B2, 7C6, 7B9, 5E11 and 1A8 can be utilized as CAR-T cells targeting GPC3.

### Example 5: Preparation of GPC3-CAR-T Cells

In the present invention, anti-GPC3 antibody-based GPC3-CAR-T cells were prepared by transfecting T cells with the GPC3-CAR lentiviral vector prepared in Example 4 above.

Specifically, as shown in the schematic diagrams shown in FIGS. 5 and 6, after isolating peripheral blood mononuclear cells (PBMC) from blood, T cells were activated using T cell activation beads (Miltenyl Biotec, cat #130-091-441). GPC3-CAR lentivirus prepared in Example 4 was transduced into the activated T cells to prepare GPC3-CAR-T cells.

The GPC3 peptide binding ability of GPC3-CAR-T cells was confirmed by flow cytometry. The GPC3-CAR-T cells (10E9, 8B2, 7B9, 5E11 and 1A8) prepared above were tested using anti-CD3, anti-CD4, and anti-CD8 antibodies to generate CD3-, CD4-, or CD8-activated GPC3-CAR-T cells. After sorting into cells, reacting with the FITC-GPC3 peptide, fluorescence intensity was measured using a flow cytometer.

As a result, as shown in FIG. 7, it was confirmed that all CD3-, CD4-, or CD8-activated GPC3-CAR-T cells bound to the GPC3 peptide.

### Example 6: Confirmation of GPC3-CAR-T Cell Activation in GPC3 Expressing Cells

In the present invention, in order to confirm that the GPC3-CAR-T cells prepared in Example 5 were specifically activated by GPC3-expressing cells, the degree of IFNy secretion by GPC3-CAR-T cells was confirmed in the presence of target cells.

As target cells, A549 cells (human lung adenocarcinoma cell line; ATCC® CCL-185TM) that did not express GPC3 and HepG2 cells (human liver cancer cell line; ATCC® HB-8065TM) that expressed GPC3 were used, Target cells and GPC3-CAR-T cells were reacted at a ratio of 1:2, 1:1 or 1:0.5 for a certain period of time, then stained with surface & intra antibody and measured by flow cytometry (INF-r, CD4, CD8 staining).

As a result, as shown in FIGS. 8a to 8e, T cells were not activated in A549 cells that did not express GPC3, whereas T cells were activated in HepG2 cells that expressed GPC3. It was confirmed that IFNy secretion was increased in all of CD4-or CD8-activated GPC3-CAR-T cells.

### Example 7: Confirmation of the Killing Effect of GPC3-CAR-T Cells on GPC3-Expressing Cells

In the present invention, the killing effect of target cells by anti-GPC3 antibody-based GPC3-CAR-T cells was confirmed.

As target cells, A549 cells (human lung adenocarcinoma cell line; ATCC® CCL-185TM) that did not express GPC3 and HepG2 cells (human liver cancer cell line; ATCC® HB-8065TM) that expressed GPC3 were used, GPC3-CAR-T cells were mixed at ratios of 1:20, 1:10, 1:4, 1:2, and 1:1, respectively, and cultured for 8 hours, followed by luminescence (CytoTox-Glo Cytotoxicity Assay, Promega, cat. NO G9291) was measured. The degree of cell apoptosis was calculated from the measured values using the following Equation 1.

[Equation 1]

% Cytotoxicity=[(Experimental-Effector Spontaneous-Target Spontaneous)/(Target Maximum-Target Spontaneous)]×100

Experimental: Luminescence value derived from target cell and CAR-T cell composite culture medium

Effector Spontaneous: Luminescence value derived from CAR-T cell-only medium

Target Spontaneous: Luminescence value derived from target cell-only medium

Target Maximum: Luminescence value derived from 100% lysis (using lysis reagent) of target cells

As a result, as shown in FIG. 9, it was confirmed that the anti-GPC3 antibody-based GPC3-CAR-T cells specifically killed HepG2 cells expressing GPC3.

### INDUSTRIAL APPLICABILITY

It was confirmed that six types of GPC3-specific antibodies (10E9, 8B2, 7C6, 7B9, 5E11, 1A8) selected in the present invention specifically bound to the GPC3 antigen, and it is possible to produce a chimeric antigen receptor (CAR) and CAR-T that target GPC3 using the established antibody. In addition, since it was confirmed that the GPC3-CAR-T cell prepared in the present invention activated the CAR-T cell in the presence of the GPC3 antigen and effectively killed cells expressing GPC3, the present GPC3-specific antibodies and chimeric antigen receptors & CAR-T cells prepared by the above antibody can be applied for use in preventing or treating a cancer or tumor expressing GPC3.

1. An antibody specifically binding to GPC3 or a fragment thereof, comprising:
(1) a heavy chain variable region comprising a CDR1 region represented by the amino acids of SEQ ID NO: 1, a CDR2 region represented by the amino acids of SEQ ID NO: 2, and a CDR3 region represented by the amino acids of SEQ ID NO: 3, and a light chain variable region comprising a CDR1 region including a CDR1 region represented by the amino acids of SEQ ID NO: 4, a CDR2 region represented by the amino acids of SEQ ID NO: 5, and a CDR3 region represented by the amino acids of SEQ ID NO: 6;
(2) a heavy chain variable region comprising a CDR1 region represented by the amino acids of SEQ ID NO: 11, a CDR2 region represented by the amino acids of SEQ ID NO: 12, and a CDR3 region represented by the amino acids of SEQ ID NO: 13, and a light chain variable region comprising a CDR1 region including a CDR1 region represented by the amino acids of SEQ ID NO: 14, a CDR2 region represented by the amino acids of SEQ ID NO: 15, and a CDR3 region represented by the amino acids of SEQ ID NO: 16;
(3) a heavy chain variable region comprising a CDR1 region represented by the amino acids of SEQ ID NO: 21, a CDR2 region represented by the amino acids of SEQ ID NO: 22, and a CDR3 region represented by the amino acids of SEQ ID NO: 23, and a light chain variable region comprising a CDR1 region including a CDR1 region represented by the amino acids of SEQ ID NO: 24, a CDR2 region represented by the amino acids of SEQ ID NO: 25, and a CDR3 region represented by the amino acids of SEQ ID NO: 26;
(4) a heavy chain variable region comprising a CDR1 region represented by the amino acids of SEQ ID NO: 31, a CDR2 region represented by the amino acids of SEQ ID NO: 32, and a CDR3 region represented by the amino acids of SEQ ID NO: 33, and a light chain variable region comprising a CDR1 region including a CDR1 region represented by the amino acids of SEQ ID NO: 34, a CDR2 region represented by the amino acids of SEQ ID NO: 35, and a CDR3 region represented by the amino acids of SEQ ID NO: 36;
(5) a heavy chain variable region comprising a CDR1 region represented by the amino acids of SEQ ID NO: 41, a CDR2 region represented by the amino acids of SEQ ID NO: 42, and a CDR3 region represented by the amino acids of SEQ ID NO: 43, and a light chain variable region comprising a CDR1 region including a CDR1 region represented by the amino acids of SEQ ID NO: 44, a CDR2 region represented by the amino acids of SEQ ID NO: 45, and a CDR3 region represented by the amino acids of SEQ ID NO: 46; or
(6) a heavy chain variable region comprising a CDR1 region represented by the amino acids of SEQ ID NO: 51, a CDR2 region represented by the amino acids of SEQ ID NO: 52, and a CDR3 region represented by the amino acids of SEQ ID NO: 53, and a light chain variable region comprising a CDR1 region including a CDR1 region represented by the amino acids of SEQ ID NO: 54, a CDR2 region represented by the amino acids of SEQ ID NO: 55, and a CDR3 region represented by the amino acids of SEQ ID NO: 56.

2. The antibody specifically binding to GPC3 or a fragment thereof of claim 1,
wherein the (1) antibody comprises a heavy chain variable region represented by amino acids of SEQ ID NO: 7 and a light chain variable region represented by amino acids of SEQ ID NO: 8,
the (2) antibody comprises a heavy chain variable region represented by amino acids of SEQ ID NO: 17 and a light chain variable region represented by amino acids of SEQ ID NO: 18,
the (3) antibody comprises a heavy chain variable region represented by amino acids of SEQ ID NO: 27 and a light chain variable region represented by amino acids of SEQ ID NO: 28,
the (4) antibody comprises a heavy chain variable region represented by amino acids of SEQ ID NO: 37 and a light chain variable region represented by amino acids of SEQ ID NO: 38,
the (5) antibody comprises a heavy chain variable region represented by amino acids of SEQ ID NO: 47 and a light chain variable region represented by amino acids of SEQ ID NO: 48; or
the (6) antibody comprises a heavy chain variable region represented by amino acids of SEQ ID NO: 57 and a light chain variable region represented by amino acids of SEQ ID NO: 58.

3. A polynucleotide encoding an antibody specifically binding to GPC3 or a fragment thereof of claim 1.

4. A vector comprising the polynucleotide encoding the antibody specifically binding to GPC3 or a fragment thereof of claim 1.

5. A recombinant cell producing the antibody or fragment thereof specifically binding to GPC3 or a fragment thereof transformed with the vector of claim 4.

6. A chimeric antigen receptor (CAR) comprising: a GPC3-binding domain; a transmembrane domain; a costimulatory domain; and an intracellular signal transduction domain,
wherein the GPC3-binding domain is an antibody specifically binding to GPC3 or a fragment thereof, comprising:
(1) a heavy chain variable region comprising a CDR1 region represented by the amino acids of SEQ ID NO: 1, a CDR2 region represented by the amino acids of SEQ ID NO: 2, and a CDR3 region represented by the amino acids of SEQ ID NO: 3, and a light chain variable region comprising a CDR1 region including a CDR1 region represented by the amino acids of SEQ ID NO: 4, a CDR2 region represented by the amino acids of SEQ ID NO: 5, and a CDR3 region represented by the amino acids of SEQ ID NO: 6;
(2) a heavy chain variable region comprising a CDR1 region represented by the amino acids of SEQ ID NO: 11, a CDR2 region represented by the amino acids of SEQ ID NO: 12, and a CDR3 region represented by the amino acids of SEQ ID NO: 13, and a light chain variable region comprising a CDR1 region including a CDR1 region represented by the amino acids of SEQ ID NO: 14, a CDR2 region represented by the amino acids of SEQ ID NO: 15, and a CDR3 region represented by the amino acids of SEQ ID NO: 16;
(3) a heavy chain variable region comprising a CDR1 region represented by the amino acids of SEQ ID NO: 21, a CDR2 region represented by the amino acids of SEQ ID NO: 22, and a CDR3 region represented by the amino acids of SEQ ID NO: 23, and a light chain variable region comprising a CDR1 region including a CDR1 region represented by the amino acids of SEQ ID NO: 24, a CDR2 region represented by the amino acids of SEQ ID NO: 25, and a CDR3 region represented by the amino acids of SEQ ID NO: 26;
(4) a heavy chain variable region comprising a CDR1 region represented by the amino acids of SEQ ID NO: 31, a CDR2 region represented by the amino acids of SEQ ID NO: 32, and a CDR3 region represented by the amino acids of SEQ ID NO: 33, and a light chain variable region comprising a CDR1 region including a CDR1 region represented by the amino acids of SEQ ID NO: 34, a CDR2 region represented by the amino acids of SEQ ID NO: 35, and a CDR3 region represented by the amino acids of SEQ ID NO: 36;
(5) a heavy chain variable region comprising a CDR1 region represented by the amino acids of SEQ ID NO: 41, a CDR2 region represented by the amino acids of SEQ ID NO: 42, and a CDR3 region represented by the amino acids of SEQ ID NO: 43, and a light chain variable region comprising a CDR1 region including a CDR1 region represented by the amino acids of SEQ ID NO: 44, a CDR2 region represented by the amino acids of SEQ ID NO: 45, and a CDR3 region represented by the amino acids of SEQ ID NO: 46; or
(6) a heavy chain variable region comprising a CDR1 region represented by the amino acids of SEQ ID NO: 51, a CDR2 region represented by the amino acids of SEQ ID NO: 52, and a CDR3 region represented by the amino acids of SEQ ID NO: 53, and a light chain variable region comprising a CDR1 region including a CDR1 region represented by the amino acids of SEQ ID NO: 54, a CDR2 region represented by the amino acids of SEQ ID NO: 55, and a CDR3 region represented by the amino acids of SEQ ID NO: 56.

7. The chimeric antigen receptor of claim 6, wherein the transmembrane domain is a protein selected from the group consisting of CD8a, CD4, CD28, CD137, CD80, CD86, CD152 and PD1,
the costimulatory domain is a protein selected from the group consisting of CD28, 4-1BB, OX-40 and ICOS, and
the intracellular signal transduction domain is CD3.

8. The chimeric antigen receptor of claim 6, further comprising a hinge region between a C-terminus of the GPC3-binding domain and an N-terminus of the transmembrane domain.

9. A polynucleotide encoding the chimeric antigen receptor of claim 6.

10. A vector comprising the polynucleotide encoding the chimeric antigen receptor of claim 6.

11. An immune effector cell comprising: the vector of claim 10.

12. A pharmaceutical composition for use in preventing or treating a cancer or tumor expressing GPC3, comprising: the antibody specifically binding to GPC3 or the fragment thereof of claim 1 and pharmaceutically acceptable carrier.

13. The pharmaceutical composition for use according to claim 12, wherein the cancer or tumor is selected from the group consisting of liver cancer, hepatocellular cancer, gastric cancer, breast cancer, lung cancer, ovarian cancer, bronchial cancer, nasopharyngeal cancer, laryngeal cancer, pancreatic cancer, bladder cancer, colorectal cancer, colon cancer, pancreatic cancer, cervical cancer, brain cancer, Prostate cancer, bone cancer, skin cancer, thyroid cancer, parathyroid cancer, kidney cancer, esophageal cancer, biliary tract cancer, testicular cancer, rectal cancer, head and neck cancer, cervical cancer, ureter cancer, osteosarcoma, neuroblastoma, melanoma, fibrosarcoma, rhabdomyosarcoma, astrocytoma, neuroblastoma and glioma.

14. An immune effector cell comprising the polynucleotide encoding the chimeric antigen receptor of claim 6.

15. A pharmaceutical composition for use in preventing or treating a cancer or tumor expressing GPC3, comprising the immune effector cell of claim 11 and a pharmaceutically acceptable carrier.

16. A pharmaceutical composition for use in preventing or treating a cancer or tumor expressing GPC3, comprising the immune effector cell of claim 14 and a pharmaceutically acceptable carrier.

17. A method for preventing or treating a cancer or tumor expressing GPC3, comprising administering to a subject in need thereof the pharmaceutical composition of claim 12.

18. A method for preventing or treating a cancer or tumor expressing GPC3, comprising administering to a subject in need thereof the pharmaceutical composition of claim 15.

19. A method for preventing or treating a cancer or tumor expressing GPC3, comprising administering to a subject in need thereof the pharmaceutical composition of claim 16.


SEQUENCE LISTING (86 sequences)

SEQ ID NO: 1 - protein, 8 aa, Artificial
  10E9_VH_CDR1
GYTFTSYW

SEQ ID NO: 2 - protein, 8 aa, Artificial
  10E9_VH_CDR2
IYPGNSDT

SEQ ID NO: 3 - protein, 11 aa, Artificial
  10E9_VH_CDR3
TRGEYDYVLAY

SEQ ID NO: 4 - protein, 6 aa, Artificial
  10E9_VL_CDR1
QSISNN

SEQ ID NO: 5 - protein, 3 aa, Artificial
  10E9_VL_CDR2
FAS

SEQ ID NO: 6 - protein, 11 aa, Artificial
  10E9_VL_CDR3
QQSNSWPHMYT

SEQ ID NO: 7 - protein, 118 aa, Artificial
  10E9_VH
EVQLEESGTVLARPGASVKMSCKASGYTFTSYWMHWIKQRPGQGLEWIGAIYPGNSDTSYNQKFKGKAKLTAVTSTSTAYMELSSLTNEDSAVYYCTRGEYDYVLAYWGQGTLVTVSA

SEQ ID NO: 8 - protein, 109 aa, Artificial
  10E9_VL
DIVMTQSPATLSVTPGDSVSLSCRASQSISNNLHWYQQKSHESPRLLIKFASQSISGIPSRFSGSGSGTDFTLSINSVETEDFGMYFCQQSNSWPHMYTFGGGTKLEIK

SEQ ID NO: 9 - DNA, 354 bp, Artificial
  10E9_VH
gaggtgcagctggaggagtctgggactgtgctggcaaggcctggggcttcagtgaagatgtcctgcaaggcttctggctacacctttaccagctactggatgcactggataaaacagaggcctggacagggtctggaatggattggcgctatttatcctggaaatagtgatactagctacaaccagaagttcaagggcaaggccaaactgactgcagtcacatccaccagcactgcctacatggagctcagcagcctgacaaatgaggactctgcggtctattactgtacaagaggggaatatgattacgtccttgcttactggggccaagggactctggtcactgtctctgca

SEQ ID NO: 10 - DNA, 327 bp, Artificial
  10E9_VL
gacattgtgatgacccagtctccagccaccctgtctgtgactccaggagatagcgtcagtctttcctgcagggccagccaaagtattagcaacaacctacactggtatcaacaaaaatcacatgagtctccaaggcttctcatcaagtttgcttcccagtccatctctgggatcccctccaggttcagtggcagtggatcagggacagatttcactctcagtatcaacagtgtggagactgaagattttggaatgtatttctgtcaacagagtaacagctggcctcacatgtacacgttcggaggggggaccaagctggaaataaaa

SEQ ID NO: 11 - protein, 8 aa, Artificial
  8B2_VH_CDR1
GYTFTSYN

SEQ ID NO: 12 - protein, 8 aa, Artificial
  8B2_VH_CDR2
IYPGNGYT

SEQ ID NO: 13 - protein, 9 aa, Artificial
  8B2_VH_CDR3
ARGGGPFAY

SEQ ID NO: 14 - protein, 6 aa, Artificial
  8B2_VL_CDR1
QDVGTA

SEQ ID NO: 15 - protein, 3 aa, Artificial
  8B2_VL_CDR2
WPS

SEQ ID NO: 16 - protein, 9 aa, Artificial
  8B2_VL_CDR3
QQYSSYPFT

SEQ ID NO: 17 - protein, 116 aa, Artificial
  8B2_VH
QVQLQQPGAELVKPGASVKMSCKASGYTFTSYNMHWVKQTPGQGLEWIGAIYPGNGYTSYNQKFKGKATLTADKSSSTAYMQLSSLTSEDSAVYYCARGGGPFAYWGQGTLVTVSA

SEQ ID NO: 18 - protein, 107 aa, Artificial
  8B2_VL
DIVMTQSHKFMSTSVGDRVSITCKASQDVGTAVAWYQQKPGQSPKLLIYWPSTRHTGVPDRFTGSGSGTDFTLTISNVQSEDLADYFCQQYSSYPFTFGSGTKLEIK

SEQ ID NO: 19 - protein, 348 aa, Artificial
  8B2_VH
CAGGTCCAACTGCAGCAGCCTGGGGCTGAGCTGGTGAAGCCTGGGGCCTCAGTGAAGATGTCCTGCAAGGCTTCTGGCTACACATTTACCAGTTACAATATGCACTGGGTAAAGCAGACACCTGGACAGGGCCTGGAATGGATTGGAGCTATTTATCCAGGAAATGGTTATACTTCCTACAATCAGAAGTTCAAAGGCAAGGCCACATTGACTGCAGACAAATCCTCCAGCACAGCCTACATGCAGCTCAGCAGCCTGACATCTGAGGACTCTGCGGTCTATTACTGTGCAAGAGGGGGTGGGCCGTTTGCTTACTGGGGCCAAGGGACTCTGGTCACTGTCTCTGCA

SEQ ID NO: 20 - DNA, 321 bp, Artificial
  8B2_VL
gacattgtgatgacccagtctcacaaattcatgtccacatcagtaggagacagggtcagcatcacctgcaaggccagtcaggatgtgggtactgctgtagcctggtatcaacagaaaccagggcaatctcctaaactactgatttactggccatccacccggcacactggagtccctgatcgcttcacaggcagtggatctgggacagatttcactctcaccattagcaatgtgcagtctgaagacttggcagattatttctgtcagcaatatagcagctatccattcacgttcggctcggggacaaagttggaaataaaa

SEQ ID NO: 21 - protein, 8 aa, Artificial
  7C6_VH_CDR1
GYTFTTYY

SEQ ID NO: 22 - protein, 8 aa, Artificial
  7C6_VH_CDR2
INPSNGGT

SEQ ID NO: 23 - protein, 5 aa, Artificial
  7C6_VH_CDR3
TTFAY

SEQ ID NO: 24 - protein, 6 aa, Artificial
  7C6_VL_CDR1
QDINKY

SEQ ID NO: 25 - protein, 3 aa, Artificial
  7C6_VL_CDR2
YTS

SEQ ID NO: 26 - protein, 8 aa, Artificial
  7C6_VL_CDR3
LQYDNLWT

SEQ ID NO: 27 - protein, 112 aa, Artificial
  7C6_VH
QVQLQQPGAELVKPGASVKLSCKASGYTFTTYYMYWVKQRPGQGLEWIGEINPSNGGTNFNEKFKSKATLTVDKSSSTAYMQLSSLTSEDSAVYYCTTFAYWGQGTLVTVSA

SEQ ID NO: 28 - protein, 106 aa, Artificial
  7C6_VL
DIVMTQSPSSLSASLGGKVTITCKASQDINKYIVWYQHKPGKGPRLLIHYTSILQPGIPSRFSGSGSGRDYSFSISNLEPEDIATYYCLQYDNLWTFGGGTKLEIK

SEQ ID NO: 29 - DNA, 336 bp, Artificial
  7C6_VH
caggtccaactgcagcagcctggggctgaactggtgaagcctggggcttcagtgaagttgtcctgcaaggcttctggctacaccttcaccacctactatatgtactgggtgaagcagaggcctggacaaggccttgagtggattggagagattaatcctagcaatggtggtactaacttcaatgagaagttcaagagcaaggccacactgacggtagacaaatcctccagcacagcatacatgcaactcagcagcctgacatctgaggactctgcggtctattactgtacgacgtttgcttactggggccaagggactctggtcactgtctctgca

SEQ ID NO: 30 - DNA, 318 bp, Artificial
  7C6_VL
gacattgtgatgacccagtctccatcctcactgtctgcatctctgggaggcaaagtcaccatcacttgcaaggcaagccaagacattaacaagtatatagtttggtaccaacacaagcctggaaaaggtcctaggctgctcatacattacacatctatattacagccaggcatcccatcaaggttcagtggaagtgggtctgggagagattattccttcagcatcagcaacctggagcctgaagatattgcaacttattattgtctacagtatgataatctttggacgttcggtggaggcaccaagctggaaatcaaa

SEQ ID NO: 31 - protein, 8 aa, Artificial
  7B9_VH_CDR1
GYTFTNYW

SEQ ID NO: 32 - protein, 8 aa, Artificial
  7B9_VH_CDR2
IYPGSGST

SEQ ID NO: 33 - protein, 11 aa, Artificial
  7B9_VH_CDR3
TRGDYDAKFAY

SEQ ID NO: 34 - protein, 7 aa, Artificial
  7B9_VL_CDR1
SSVSSSY

SEQ ID NO: 35 - protein, 3 aa, Artificial
  7B9_VL_CDR2
STS

SEQ ID NO: 36 - protein, 10 aa, Artificial
  7B9_VL_CDR3
QQYSGYPLIT

SEQ ID NO: 37 - protein, 118 aa, Artificial
  7B9_VH
QVQLQQPGSELVRPGASVKLSCKASGYTFTNYWMHWVKQRPGQGLEWIGNIYPGSGSTNYDEKFKSKATLTVDTSSSTAYMQLSSLTSEDSAVYYCTRGDYDAKFAYWGQGTLVTVSA

SEQ ID NO: 38 - protein, 109 aa, Artificial
  7B9_VL
DIVMTQSPAIMSASPGEKVTMTCRASSSVSSSYLHWYQQKSGASPKLWIYSTSKLASGVPARFSGSGSGTSYSLTISSVEAEDAATYYCQQYSGYPLITFGAGTKVELK

SEQ ID NO: 39 - DNA, 354 bp, Artificial
  7B9_VH
caggtccaactgcagcagcctgggtctgagctggtgaggcctggagcttcagtgaagctgtcctgcaaggcttctggctacacattcaccaactactggatgcactgggtgaaacagaggcctggacaaggccttgagtggattggaaatatttatcctggtagtggtagtactaactacgatgagaagttcaagagcaaggccacactgactgtagacacatcctccagcacagcctacatgcagctcagcagcctgacatctgaggactctgcggtctattactgtacaagaggggattacgacgcaaagtttgcttactggggccaagggactctggtcactgtctctgca

SEQ ID NO: 40 - DNA, 327 bp, Artificial
  7B9_VL
gacattgtgatgacccagtctccagcaatcatgtctgcatctccaggggaaaaggtcaccatgacctgcagggccagctcaagtgtaagttccagttacttgcactggtaccagcagaagtcaggtgcctcccccaaactctggatttatagcacatccaagttggcttctggagtccctgctcgcttcagtggcagtgggtctgggacctcttactctctcacaatcagcagtgtggaggctgaagatgctgccacttattactgccagcagtacagtggttacccactcatcacgttcggtgctgggaccaaggtggagctgaaa

SEQ ID NO: 41 - protein, 8 aa, Artificial
  5E11_VH_CDR1
GYTFSRYW

SEQ ID NO: 42 - protein, 8 aa, Artificial
  5E11_VH_CDR2
ILPGSGST

SEQ ID NO: 43 - protein, 12 aa, Artificial
  5E11_VH_CDR3
ARSARATYYFDY

SEQ ID NO: 44 - protein, 6 aa, Artificial
  5E11_VL_CDR1
QDISNY

SEQ ID NO: 45 - protein, 3 aa, Artificial
  5E11_VL_CDR2
YTS

SEQ ID NO: 46 - protein, 9 aa, Artificial
  5E11_VL_CDR3
QQGNALPYT

SEQ ID NO: 47 - protein, 119 aa, Artificial
  5E11_VH
QVQLKQSGAELMKPGASVKISCKATGYTFSRYWIEWVKQRPGHGLEWIGEILPGSGSTSYNEKFKGKATFTADTSSNTAYMQLSSLTSEDSAVYYCARSARATYYFDYWGQGTTLTVSS

SEQ ID NO: 48 - protein, 107 aa, Artificial
  5E11_VL
DIQMTQTTSSLSASLGDRVTISCWASQDISNYLNWYQQKPDGTVKLLIYYTSTLHSGVPSRFSGSGSGTDYSLTISNLEQEDIATYFCQQGNALPYTFGGGTKLEIK

SEQ ID NO: 49 - DNA, 357 bp, Artificial
  5E11_VH
caggtgcagctgaagcagtctggagctgagctgatgaagcctggggcctcagtgaagatatcctgcaaggctactggctacacattcagtaggtactggatagagtgggtaaagcagaggcctggacatggccttgagtggattggagagattttacctggaagtggtagtactagttacaatgagaagttcaagggcaaggccacattcactgcagatacatcctccaacacagcctacatgcaactcagcagcctgacatctgaggactctgccgtctattactgtgcaagatcagctcgggctacgtactactttgactactggggccaaggcaccactctcacagtctcctca

SEQ ID NO: 50 - DNA, 321 bp, Artificial
  5E11_VL
gatatccagatgacacagactacatcctccctgtctgcctctctgggagacagagtcaccatcagttgctgggcaagtcaggacattagcaattatttaaactggtatcagcagaaaccagatggaactgttaaactcctgatctactacacatcaacattacactcaggagtcccatcaaggttcagtggcagtgggtctggaacagattattctctcaccattagcaacctggagcaagaagatattgccacttacttttgccaacagggtaatgcgcttccgtacacgttcggaggggggaccaagctggaaataaaa

SEQ ID NO: 51 - protein, 8 aa, Artificial
  1A8_VH_CDR1
GYTFTSYW

SEQ ID NO: 52 - protein, 8 aa, Artificial
  1A8_VH_CDR2
IYPGSGST

SEQ ID NO: 53 - protein, 11 aa, Artificial
  1A8_VH_CDR3
TRGHYDYAMDY

SEQ ID NO: 54 - protein, 6 aa, Artificial
  1A8_VL_CDR1
QNINVW

SEQ ID NO: 55 - protein, 3 aa, Artificial
  1A8_VL_CDR2
KTS

SEQ ID NO: 56 - protein, 9 aa, Artificial
  1A8_VL_CDR3
QQGQSYPWT

SEQ ID NO: 57 - protein, 118 aa, Artificial
  1A8_VH
QVQLQQPGSELVRPGASVKLSCKASGYTFTSYWMHWVKQRHGQGLEWIGNIYPGSGSTNYDEKFKSKGTLTVDTSSRTAYMHLSSLTSEDSAVYYCTRGHYDYAMDYWGQGTSVTVSS

SEQ ID NO: 58 - protein, 107 aa, Artificial
  1A8_VL
DIVMTQSPSSLSASLGDTITITCHASQNINVWLSWYQQKPGNIPKLLIYKTSNLHTGVPSRFSGSGSGTGFTLTISSLQPEDIATYYCQQGQSYPWTFGGGTKLEIK

SEQ ID NO: 59 - DNA, 354 bp, Artificial
  1A8_VH
caggtccaactgcagcagcctgggtctgagctggtgaggcctggagcttcagtgaagctgtcctgcaaggcttctggctacacattcaccagctactggatgcactgggtgaagcagaggcatggacaaggccttgagtggattggaaatatttatcctggtagtggtagtactaactacgatgagaagttcaagagcaagggcacactgactgtagacacatcctccagaacagcctacatgcacctcagcagcctgacatctgaggactctgcggtctattactgtacaaggggtcactacgactatgctatggactactggggtcaaggaacctcagtcaccgtctcctca

SEQ ID NO: 60 - DNA, 321 bp, Artificial
  1A8_VL
gacattgtgatgacccagtctccatccagtctgtctgcatcccttggagacacaattaccatcacttgccatgccagtcagaacattaatgtttggttaagctggtaccagcagaaaccaggaaatattcctaaactattgatctataagacttccaacttgcacacaggcgtcccatcaaggtttagtggcagtggatctggaacaggtttcacattaaccatcagcagcctgcagcctgaagacattgccacttactactgtcaacagggtcaaagttatccgtggacgttcggtggaggcaccaagctggaaatcaaa

SEQ ID NO: 61 - protein, 15 aa, Artificial
  Linker
GGGGSGGGGSGGGGS

SEQ ID NO: 62 - DNA, 45 bp, Artificial
  Linker
ggtggtggtggttcgggtggtggtggttcgggtggtggtggttcg

SEQ ID NO: 63 - protein, 242 aa, Artificial
  10E9_VL-Linker-VH
DIVMTQSPATLSVTPGDSVSLSCRASQSISNNLHWYQQKSHESPRLLIKFASQSISGIPSRFSGSGSGTDFTLSINSVETEDFGMYFCQQSNSWPHMYTFGGGTKLEIKGGGGSGGGGSGGGGSEVQLEESGTVLARPGASVKMSCKASGYTFTSYWMHWIKQRPGQGLEWIGAIYPGNSDTSYNQKFKGKAKLTAVTSTSTAYMELSSLTNEDSAVYYCTRGEYDYVLAYWGQGTLVTVSA

SEQ ID NO: 64 - DNA, 726 bp, Artificial
  10E9_VL-Linker-VH
gacattgtgatgacccagtctccagccaccctgtctgtgactccaggagatagcgtcagtctttcctgcagggccagccaaagtattagcaacaacctacactggtatcaacaaaaatcacatgagtctccaaggcttctcatcaagtttgcttcccagtccatctctgggatcccctccaggttcagtggcagtggatcagggacagatttcactctcagtatcaacagtgtggagactgaagattttggaatgtatttctgtcaacagagtaacagctggcctcacatgtacacgttcggaggggggaccaagctggaaataaaaggtggtggtggttcgggtggtggtggttcgggtggtggtggttcggaggtgcagctggaggagtctgggactgtgctggcaaggcctggggcttcagtgaagatgtcctgcaaggcttctggctacacctttaccagctactggatgcactggataaaacagaggcctggacagggtctggaatggattggcgctatttatcctggaaatagtgatactagctacaaccagaagttcaagggcaaggccaaactgactgcagtcacatccaccagcactgcctacatggagctcagcagcctgacaaatgaggactctgcggtctattactgtacaagaggggaatatgattacgtccttgcttactggggccaagggactctggtcactgtctctgca

SEQ ID NO: 65 - protein, 238 aa, Artificial
  8B2_VL-Linker-VH
DIVMTQSHKFMSTSVGDRVSITCKASQDVGTAVAWYQQKPGQSPKLLIYWPSTRHTGVPDRFTGSGSGTDFTLTISNVQSEDLADYFCQQYSSYPFTFGSGTKLEIKGGGGSGGGGSGGGGSQVQLQQPGAELVKPGASVKMSCKASGYTFTSYNMHWVKQTPGQGLEWIGAIYPGNGYTSYNQKFKGKATLTADKSSSTAYMQLSSLTSEDSAVYYCARGGGPFAYWGQGTLVTVSA

SEQ ID NO: 66 - DNA, 714 bp, Artificial
  8B2_VL-Linker-VH
gacattgtgatgacccagtctcacaaattcatgtccacatcagtaggagacagggtcagcatcacctgcaaggccagtcaggatgtgggtactgctgtagcctggtatcaacagaaaccagggcaatctcctaaactactgatttactggccatccacccggcacactggagtccctgatcgcttcacaggcagtggatctgggacagatttcactctcaccattagcaatgtgcagtctgaagacttggcagattatttctgtcagcaatatagcagctatccattcacgttcggctcggggacaaagttggaaataaaaggtggtggtggttcgggtggtggtggttcgggtggtggtggttcgcaggtccaactgcagcagcctggggctgagctggtgaagcctggggcctcagtgaagatgtcctgcaaggcttctggctacacatttaccagttacaatatgcactgggtaaagcagacacctggacagggcctggaatggattggagctatttatccaggaaatggttatacttcctacaatcagaagttcaaaggcaaggccacattgactgcagacaaatcctccagcacagcctacatgcagctcagcagcctgacatctgaggactctgcggtctattactgtgcaagagggggtgggccgtttgcttactggggccaagggactctggtcactgtctctgca

SEQ ID NO: 67 - protein, 233 aa, Artificial
  7C6_VL-Linker-VH
DIVMTQSPSSLSASLGGKVTITCKASQDINKYIVWYQHKPGKGPRLLIHYTSILQPGIPSRFSGSGSGRDYSFSISNLEPEDIATYYCLQYDNLWTFGGGTKLEIKGGGGSGGGGSGGGGSQVQLQQPGAELVKPGASVKLSCKASGYTFTTYYMYWVKQRPGQGLEWIGEINPSNGGTNFNEKFKSKATLTVDKSSSTAYMQLSSLTSEDSAVYYCTTFAYWGQGTLVTVSA

SEQ ID NO: 68 - DNA, 699 bp, Artificial
  7C6_VL-Linker-VH
gacattgtgatgacccagtctccatcctcactgtctgcatctctgggaggcaaagtcaccatcacttgcaaggcaagccaagacattaacaagtatatagtttggtaccaacacaagcctggaaaaggtcctaggctgctcatacattacacatctatattacagccaggcatcccatcaaggttcagtggaagtgggtctgggagagattattccttcagcatcagcaacctggagcctgaagatattgcaacttattattgtctacagtatgataatctttggacgttcggtggaggcaccaagctggaaatcaaaggtggtggtggttcgggtggtggtggttcgggtggtggtggttcgcaggtccaactgcagcagcctggggctgaactggtgaagcctggggcttcagtgaagttgtcctgcaaggcttctggctacaccttcaccacctactatatgtactgggtgaagcagaggcctggacaaggccttgagtggattggagagattaatcctagcaatggtggtactaacttcaatgagaagttcaagagcaaggccacactgacggtagacaaatcctccagcacagcatacatgcaactcagcagcctgacatctgaggactctgcggtctattactgtacgacgtttgcttactggggccaagggactctggtcactgtctctgca

SEQ ID NO: 69 - protein, 242 aa, Artificial
  7B9_VL-Linker-VH
DIVMTQSPAIMSASPGEKVTMTCRASSSVSSSYLHWYQQKSGASPKLWIYSTSKLASGVPARFSGSGSGTSYSLTISSVEAEDAATYYCQQYSGYPLITFGAGTKVELKGGGGSGGGGSGGGGSQVQLQQPGSELVRPGASVKLSCKASGYTFTNYWMHWVKQRPGQGLEWIGNIYPGSGSTNYDEKFKSKATLTVDTSSSTAYMQLSSLTSEDSAVYYCTRGDYDAKFAYWGQGTLVTVSA

SEQ ID NO: 70 - DNA, 726 bp, Artificial
  7B9_VL-Linker-VH
gacattgtgatgacccagtctccagcaatcatgtctgcatctccaggggaaaaggtcaccatgacctgcagggccagctcaagtgtaagttccagttacttgcactggtaccagcagaagtcaggtgcctcccccaaactctggatttatagcacatccaagttggcttctggagtccctgctcgcttcagtggcagtgggtctgggacctcttactctctcacaatcagcagtgtggaggctgaagatgctgccacttattactgccagcagtacagtggttacccactcatcacgttcggtgctgggaccaaggtggagctgaaaggtggtggtggttcgggtggtggtggttcgggtggtggtggttcgcaggtccaactgcagcagcctgggtctgagctggtgaggcctggagcttcagtgaagctgtcctgcaaggcttctggctacacattcaccaactactggatgcactgggtgaaacagaggcctggacaaggccttgagtggattggaaatatttatcctggtagtggtagtactaactacgatgagaagttcaagagcaaggccacactgactgtagacacatcctccagcacagcctacatgcagctcagcagcctgacatctgaggactctgcggtctattactgtacaagaggggattacgacgcaaagtttgcttactggggccaagggactctggtcactgtctctgca

SEQ ID NO: 71 - protein, 241 aa, Artificial
  5E11_VL-Linker-VH
DIQMTQTTSSLSASLGDRVTISCWASQDISNYLNWYQQKPDGTVKLLIYYTSTLHSGVPSRFSGSGSGTDYSLTISNLEQEDIATYFCQQGNALPYTFGGGTKLEIKGGGGSGGGGSGGGGSQVQLKQSGAELMKPGASVKISCKATGYTFSRYWIEWVKQRPGHGLEWIGEILPGSGSTSYNEKFKGKATFTADTSSNTAYMQLSSLTSEDSAVYYCARSARATYYFDYWGQGTTLTVSS

SEQ ID NO: 72 - DNA, 723 bp, Artificial
  5E11_VL-Linker-VH
gatatccagatgacacagactacatcctccctgtctgcctctctgggagacagagtcaccatcagttgctgggcaagtcaggacattagcaattatttaaactggtatcagcagaaaccagatggaactgttaaactcctgatctactacacatcaacattacactcaggagtcccatcaaggttcagtggcagtgggtctggaacagattattctctcaccattagcaacctggagcaagaagatattgccacttacttttgccaacagggtaatgcgcttccgtacacgttcggaggggggaccaagctggaaataaaaggtggtggtggttcgggtggtggtggttcgggtggtggtggttcgcaggtgcagctgaagcagtctggagctgagctgatgaagcctggggcctcagtgaagatatcctgcaaggctactggctacacattcagtaggtactggatagagtgggtaaagcagaggcctggacatggccttgagtggattggagagattttacctggaagtggtagtactagttacaatgagaagttcaagggcaaggccacattcactgcagatacatcctccaacacagcctacatgcaactcagcagcctgacatctgaggactctgccgtctattactgtgcaagatcagctcgggctacgtactactttgactactggggccaaggcaccactctcacagtctcctca

SEQ ID NO: 73 - protein, 240 aa, Artificial
  1A8_VL-Linker-VH
DIVMTQSPSSLSASLGDTITITCHASQNINVWLSWYQQKPGNIPKLLIYKTSNLHTGVPSRFSGSGSGTGFTLTISSLQPEDIATYYCQQGQSYPWTFGGGTKLEIKGGGGSGGGGSGGGGSQVQLQQPGSELVRPGASVKLSCKASGYTFTSYWMHWVKQRHGQGLEWIGNIYPGSGSTNYDEKFKSKGTLTVDTSSRTAYMHLSSLTSEDSAVYYCTRGHYDYAMDYWGQGTSVTVSS

SEQ ID NO: 74 - DNA, 720 bp, Artificial
  1A8_VL-Linker-VH
gacattgtgatgacccagtctccatccagtctgtctgcatcccttggagacacaattaccatcacttgccatgccagtcagaacattaatgtttggttaagctggtaccagcagaaaccaggaaatattcctaaactattgatctataagacttccaacttgcacacaggcgtcccatcaaggtttagtggcagtggatctggaacaggtttcacattaaccatcagcagcctgcagcctgaagacattgccacttactactgtcaacagggtcaaagttatccgtggacgttcggtggaggcaccaagctggaaatcaaaggtggtggtggttcgggtggtggtggttcgggtggtggtggttcgcaggtccaactgcagcagcctgggtctgagctggtgaggcctggagcttcagtgaagctgtcctgcaaggcttctggctacacattcaccagctactggatgcactgggtgaagcagaggcatggacaaggccttgagtggattggaaatatttatcctggtagtggtagtactaactacgatgagaagttcaagagcaagggcacactgactgtagacacatcctccagaacagcctacatgcacctcagcagcctgacatctgaggactctgcggtctattactgtacaaggggtcactacgactatgctatggactactggggtcaaggaacctcagtcaccgtctcctca

SEQ ID NO: 75 - DNA, 1178 bp, Artificial
  EF1 promoter
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 76 - DNA, 63 bp, Artificial
  signal peptide
atggccttaccagtgaccgccttgctcctgccgctggccttgctgctccacgccgccaggccg

SEQ ID NO: 77 - DNA, 135 bp, Artificial
  CD8 Hinge
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 78 - DNA, 72 bp, Artificial
  transmembrane domain
atctacatctgggcgcccttggccgggacttgtggggtccttctcctgtcactggttatcaccctttactgc

SEQ ID NO: 79 - DNA, 126 bp, Artificial
  4-1BB
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 80 - DNA, 336 bp, Artificial
  CD3zeta
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 81 - DNA, 591 bp, Artificial
  WPRE
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 82 - protein, 21 aa, Artificial
  signal peptide
MALPVTALLLPLALLLHAARP

SEQ ID NO: 83 - protein, 45 aa, Artificial
  CD8 Hinge
TTTPAPRPPTPAPTIASQPLSLRPEACRPAAGGAVHTRGLDFACD

SEQ ID NO: 84 - protein, 24 aa, Artificial
  transmembrane domain
IYIWAPLAGTCGVLLLSLVITLYC

SEQ ID NO: 85 - protein, 42 aa, Artificial
  4-1BB
KRGRKKLLYIFKQPFMRPVQTTQEEDGCSCRFPEEEEGGCEL

SEQ ID NO: 86 - protein, 112 aa, Artificial
  CD3zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR